(19) United States
(12) Patent Application Publication
(10) Pub. No.: US 2026/0245686 A1
(43) Pub. Date: Aug. 20, 2026

(54) MAIA Outcome Feedback Computing System

(71) Applicants: Zachary Ruhl, Littleton, CO (US); Joshua Hofstad, Berlin (DE); Anson Antony, Boston, MA (US); Eric Lau, Orland Park, IL (US); Christopher Kramer, Denver, IL (US); Owen Hulse, IV, Orland Park, IL (US)

(72) Inventors: Zachary Ruhl, Littleton, CO (US); Joshua Hofstad, Berlin (DE); Anson Antony, Boston, MA (US); Eric Lau, Orland Park, IL (US); Christopher Kramer, Denver, IL (US); Owen Hulse, IV, Orland Park, IL (US)

(73) Assignee: MAIA Medical Billing Corp., Sheridan, WY (US)

(21) Appl. No.: 19/553,903

(22) Filed: Mar. 2, 2026

(51) Int. Cl. G16H 10/60; G06F 18/21; G06F 18/213; G06N 5/04

(52) U.S. Cl. CPC G16H 10/60; G06F 18/213; G06F 18/217; G06N 5/04

(57) ABSTRACT

MAIA Outcome Feedback Computing System provides users with a real-time, document guidance, interface to upload medical claims, in multiple different formats. Document classification is performed on the uploaded data to determine a document type. Claim features are identified and extracted specific to each contextual document type, forwarded to a pre-approval and feedback manager and used to perform semantic and keyword searches on knowledge databases specific to each document type. The claims, and relevant data are input into a machine learning model, trained to predict medical billing codes using medical claims and generate: a confidence score and results summary for each billing code. Based on a comparison of the confidence score to a threshold, one of a plurality of validation processes is performed on each billing code, results are output to a user. Validated codes may be automatically submitted to third-party insurance providers, monitored for denials, and automatically appealed.

### BACKGROUND OF INVENTION

As healthcare systems implement computing systems into their practices, and integrate their practices and computing systems with the computing systems of other healthcare systems and insurance providers, data is recorded, input, stored, managed, analyzed, processed and transmitted using a plurality of different digital storage systems, protocols and formats which can lead to data errors. Additionally, computing systems and digital systems can misinterpret user inputs based on differences in the use of language, from one person to the next. Both data errors and misinterpretation errors in the identification and assignment of medical billing codes to medical claims is particularly critical, due to billing code errors directly affecting the finances of multiple entities.

### SUMMARY OF THE INVENTION

MAIA Outcome Feedback computing system is an end-to-end medical billing platform which facilitates in increasing the accuracy of, correctly assigning medical billing codes to medical data as follows:

MAIA's Outcome Feedback computing platform provides a real-time, document guidance, interactive user interface by which users upload medical claims, in a plurality of formats, from medical providers talk-to-text operative notes processed in real-time, or, claim by claim, or, as specific data structures in batch processing jobs.

Document classification is performed on the uploaded medical claims, to identify a contextual document type of the uploaded medical claims, such as, operative notes or batch processing code formatting, etc., and automatically performs a series of feature extraction, pre-approval, and feedback operations defined for each contextual document types (i.e., document processing pipelines), additionally, the extracted features are then used in coordinated, semantic and key-word database searches (i.e., hybrid retrieval process) of knowledge databases specific to the contextual document types.

The uploaded medical claims and the relevant knowledge database data are input into at least one embedding model, trained to correlate medical billing codes to medical data features, and generate and assign medical billing codes to the uploaded medical claims, which includes a confidence score for each assigned medical billing code, and a results summary for each assigned medical billing code.

Based on a comparison of at least, the confidence score and a threshold, which may be user defined, or generated automatically, each assigned medical billing code goes through at least one, data validation process. For example, a faster, data model based validation process for medical billing codes with confidence scores above a threshold, and a more complex and diligent validation process, such as forms of crowdsourcing, for medical billing codes with confidence scores below a certain threshold.

Assigned and validated medical billing codes may be submitted directly to third-party insurance providers, monitored, and, in the instance of a medical billing code denial, generate an automatic response to the denial, submit the automatically generated response to the denial to the third-party insurance provider, and use the denial feedback to adjust the medical billing code assignment and validation process.

The functionality of adjusting confidence score thresholds in the determination of the final validation processes implemented, allows for optimizing accuracy and performance in the assignment and validation of medical billing codes assigned to medical claims.

### BRIEF DESCRIPTION OF DRAWINGS

FIG. 1 is a block diagram of MAIA Outcome Feedback Computing System 100.

FIG. 2 is a block diagram of MAIA Data Store 200.

FIG. 3 is a block diagram of MAIA User Interface 300.

FIG. 4 is a block diagram of the MAIA Orchestration Engine 400.

FIG. 5 is a logical order of operations flowchart for MAIA Orchestration Engine 400.

FIG. 6 is a block diagram of the MAIA Primary LLM Engine 600.

FIG. 7 is a logical order of operations flowchart for MAIA Primary LLM Engine 600.

FIG. 8 is a block diagram of the Outcome Feeback System 800.

FIG. 9 is a logical order of operations flowchart of Outcome Feeback System 800.

FIG. 10 is a logical order of operations flowchart of Prior Authorization Manager 880.

FIG. 11 is a Real-Time Documentation Guidance Manager 380 Logical Operation Flowchart.

### DETAILED DESCRIPTION

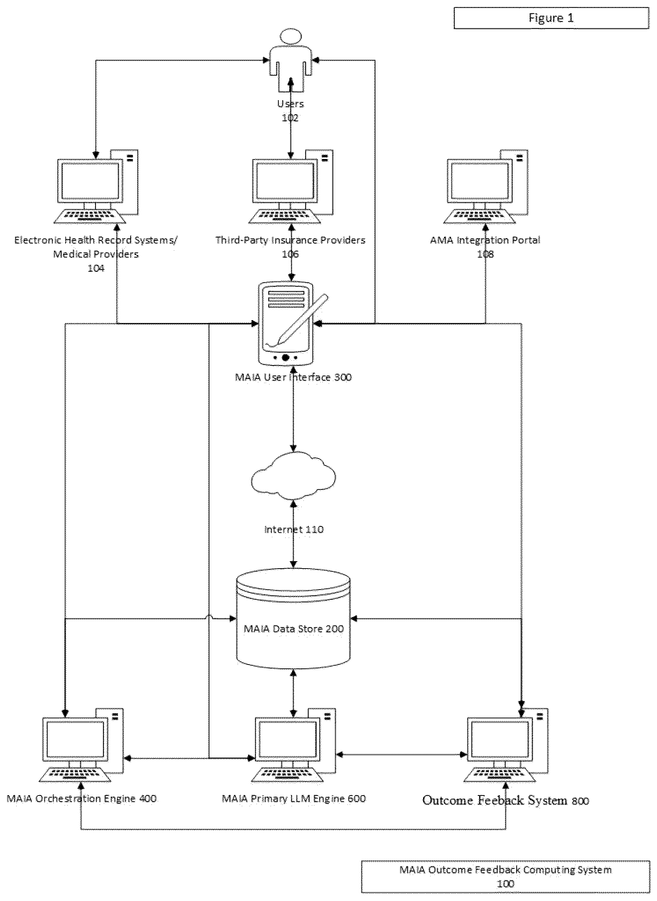

FIG. 1 illustrates MAIA Outcome Feedback Computing System 100. Users 102 login and interact with MAIA Outcome Feedback Computing System 100 via MAIA User Interface 300 operating on a computing device, such as a computer, tablet, or smartphone. Users 102 may include medical coders, billing specialists, revenue cycle management personnel, practice administrators, physicians, and compliance auditors associated with Electronic Health Record (“EHR”) Systems/Medical Providers 104, as well as claims processors and auditors associated with third-party payers, such as Third-Party Insurance Providers 106. Compliance auditors and American Medical Association staff may login and interact with MAIA Outcome Feedback Computing System via AMA Integration Portal 108.

For purposes of this Application, computing devices, are explicitly defined as comprising at least one hardware processor, coupled to at least one non-transitory computer readable medium. Computing devices may connect and exchange data with Internet 110 via wired and wireless network connections.

Although the embodiments described herein are illustrated primarily in the context of medical claims and medical billing, the disclosed systems and methods are not so limited and may be applied to any domain involving document ingestion, classification, feature extraction, knowledge retrieval, rule-based or model-based validation, and decision output generation, including but not limited to financial services, legal analysis, insurance underwriting, logistics, compliance auditing, and enterprise document automation.

MAIA User Interface 300 may operate an interactive graphical user interface (GUI) rendered on a display of a computing device via an Input/Output (I/O) interface. The I/O interface may allow Users 102 to connect, download, and upload data to MAIA Outcome Feedback Computing System 100 via a plurality of I/O devices, including network interfaces, keyboards, pointing devices, displays, and touch-screen interfaces. MAIA User Interface 300 may also expose application programming interfaces (APIs) for programmatic integration with EHR systems and practice management systems.

The interactive GUI may display a login input prompt to Users 102. As Users 102 input login data, the login data is sent to Account Manager 230 which attempts to match the login data to a MAIA User Login Data 232. Account Manager 230 may execute locally on the computing device implementing MAIA User Interface 300 or remotely as part of MAIA Data Store 200 networked with MAIA Outcome Feedback Computing System 100.

Account Manager 230 may execute user authentication procedures, including username/password validation, multi-factor authentication, single sign-on (SSO), and biometric authentication. MAIA user accounts may include User Profile Data 234 and User Type Data 236. User Type Data 236 may include medical coders, billing specialists, physicians, practice administrators, compliance auditors, and third-party payer representatives. User Profile Data 234 may include organization-specific configuration parameters, such as coding philosophy preferences (e.g., conservative or aggressive coding tolerance) and specialty-specific coding rules.

If the matched user account has upload permission, defined in User Profile Data 234 or automatically granted based on User Type Data 236, Users 102 may upload medical claim data via Medical Claim Upload Manager 350.

Medical Claim Upload Manager 350 supports a plurality of input formats and methods, including: PDF Upload 351 for scanned or electronic documents; Text Upload 352 for plain text clinical notes; Optical Character Recognition (“OCR”) 353 for image-based document conversion; EHR Integration 355 for direct extraction from electronic health record systems via HL7 FHIR, HL7 v2, or CCD/C-CDA interfaces; Voice to Text 356 for direct clinician dictation transcription; and A.I. Scribe 357 for ambient clinical documentation capture wherein patient-clinician encounters are recorded and automatically structured into clinical note format.

Real-Time Processing Monitor 354 provides status updates and solicits user feedback throughout the upload and processing workflow.

Clinical documentation may be uploaded in structured or unstructured format. Structured formats may include templated EHR extracts with discrete data fields; unstructured formats may include free-text clinical narratives, dictated operative reports, and scanned documents. Clinical documentation may be processed individually, in bulk as part of a batch process, or in real-time with continuous user feedback via Real-Time Processing Monitor 354.

The uploaded medical claim data may include patient data, the patient data may include protected health information (PHI) and personally identifiable information (PII), which may be de-identified before the medical claim data is actively stored, or may be de-identified after it is used to retrieve patient historical data from local or third-party EHR systems or databases. Both the patient data identified in the uploaded medical claim data and the retrieved patient historical data may be de-identified before the data is actively stored in MAIA Outcome Feedback Computing System 100. De-identification may implement Safe Harbor or Expert Determination methods as defined by HIPAA, or jurisdiction-specific privacy protocols such as GDPR for international deployments. Users 102 may define rules for the use/protection of patient historical data, the user-defined rules may be stored in their user account, or such data protection rules may be implemented system-wide as part of a compliance protocol. Rules regarding the storing, use and transmission of patient data may also be implemented as system-wide and part of stored Compliance Rules 254.

De-identification, anonymization, or privatization of patient data may be performed locally on the computing device implementing MAIA User Interface 300, or remotely via Privatization Manager 240 at MAIA Data Store 200, in accordance with Compliance Rules 254.

Document classification is then performed on the uploaded medical claim data, to determine a document type, and intelligently routes the uploaded medical claim data and the results of the document classification, into at least one document processing pipeline based on the determined document type. Document classification may utilize rule-based classifiers, machine learning models, or large language model (LLM) based classifiers, or a combination thereof. The document classification, intelligent routing, and data processing pipeline may be performed locally, on the computing device implementing MAIA User Interface 300, or as part of a networked system, networked with MAIA Outcome Feedback Computing System 100, such as MAIA Orchestration Engine 400, implementing Document Processing Pipeline Manager 450.

Orchestration Engine 400 may provide notification to Users 102 of the results of the document classification, and the determined document type of the uploaded medical claim data via Real-Time Processing Monitor 354 and query Users 102 for feedback which may include; user approval, user content changes and user document type changes.

The uploaded medical claim data is then intelligently routed into at least one of a plurality of data processing pipelines, corresponding to the determined document type, such as Operative Note Document Pipeline 452, E&M Note Document Pipeline 454, ICD-10 Code Pipeline 456, CPT Code Pipeline 457 and HCPCS Code Pipeline 458.

For example, if the results of the document classification, is a determination that the uploaded medical claim data is an operative note document type, the uploaded medical claim data is routed to Operative Note Document Pipeline 452. If the results of the document classification, is a determination that the uploaded medical claim data is an E&M document type, the uploaded medical claim data is routed to E&M Note Document Pipeline 454. Further, if ICD-10, CPT and/or HCPCS codes are detected, the uploaded medical claim data can additionally be routed to the corresponding coding pipeline, such as, ICD-10 Code Pipeline 456, CPT Code Pipeline 457 and HCPCS Code Pipeline 458.

Document Processing Pipeline Manager 450 then performs combinations of data transformations, data analytics and feature extraction on the uploaded medical claim data, specific to the routed document processing pipeline, to extract a set of features. The combinations of data transformations, data analytics and feature extraction performed on each document type, may be implemented as rules or policies, including user defined or system-wide, or Compliance Rules 254, defined and executed for each of the respective document processing pipeline on each respective document type.

For example, Operative Note Document Pipeline 452 may perform data transformations, data analytics, and feature extraction on the uploaded medical claim data, including extraction of procedure-related attributes, anatomical indicators, and procedural components. The extracted features are subsequently used by Hybrid Retrieval Manager 460 to perform retrieval-augmented generation, with final validation including add-on code protection, NCCI validation, and RVU sequencing.

E&M Note Document Pipeline 454 may perform feature extraction on evaluation and management documentation using specialized extraction agents operating on isolated note sections.

For example, an Encounter Boundary Detection Agent isolates current visit documentation from historical patient data to prevent inappropriate inclusion of prior encounter risk indicators in current visit assessments. A Problem Complexity Agent extracts and categorizes diagnoses from the Assessment section of the uploaded medical claim document, determining problem status (new, acute, chronic, worsening) and counting unique addressable problems. A Data Complexity Agent extracts reviewed and ordered data elements from HPI, Plan, and Results sections of the uploaded medical claim document, categorizing by data type (labs, imaging, consultations, independent interpretation) and source (internal, external). A Risk Complexity Agent analyzes the Plan section to identify current management decisions, including prescription drug management, procedures ordered, and treatment escalation indicators. A Time Agent extracts total time documentation and time-based activity descriptions when time-based E&M coding is applicable. A Combiner Agent applies the two-of-three rule to the extracted MDM component levels (problem complexity, data complexity, risk complexity) to determine the appropriate E&M code level.

Further, metadata filtering may be used to isolate relevant note sections for each extraction agent based on document structure and section headers. Additionally, E&M Note Document Pipeline 454 may implement new patient versus established patient classification based on practice management system integration or extracted demographic indicators, with validation against historical encounter data.

Code pipelines, such as, ICD-10 Code Pipeline 456, CPT Code Pipeline 457 and HCPCS Code Pipeline 458 may operate in series or in parallel with Operative Note Document Pipeline 452 or E&M Note Document Pipeline 454 to extract diagnosis codes from clinical documentation. Clinical documentation requiring both procedure codes and diagnosis codes is routed to multiple pipelines simultaneously.

For example, ICD-10 Code Pipeline 456 may perform feature extraction including: diagnostic term extraction from Assessment and Impression sections of the uploaded document claim data; clinical indicator identification (signs, symptoms, test results); laterality and anatomical specificity extraction; acuity and chronicity indicators; and causal relationship identification for manifestation codes.

For example, CPT Code Pipeline 457, feature extraction may include: primary and secondary procedure identification from the clinical and/or operative report and surgical documentation; anatomical site extraction with integrated laterality indicators (left, right, bilateral); identification of the surgical approach (open, arthroscopic, percutaneous, endoscopic, laparoscopic); enumeration of distinct procedural steps for component coding; and extraction of anesthesia type, surgical findings, clinical findings, time documentation for time-based codes, and any other extraction necessary for accurate coding.

For example, HCPCS Code Pipeline 458, feature extraction may include: identification of implants, medical devices, and grafts from the clinical narrative for supply coding; extraction of drug names, dosages, and administration routes for pharmaceutical coding; identification of durable medical equipment (DME) and related supplies; anatomical and laterality specificity related to equipment or supply application; and detection of HCPCS-specific modifiers required for non-physician services or specific payer-defined equipment reporting.

Extracted diagnostic features are mapped to candidate ICD-10-CM codes using medical ontology mapping (e.g., SNOMED-CT to ICD-10-CM crosswalks), alphabetic index term matching, and tabular list navigation. Code validation includes specificity requirement verification, excludes/includes note validation, and sequencing rule application for primary and secondary diagnoses.

The extracted features and clinical documentation are used in a hybrid data retrieval process performed by Hybrid Retrieval Manager 460. The hybrid retrieval process combines vectorized semantic search using Vector Index 296 to identify similar historical cases, relevant coding examples, and semantically related knowledge base entries based on embedding similarity and structured database searching to retrieve exact-match coding rules, modifier requirements, bundling edits, and guideline references.

The hybrid retrieval process queries knowledge bases specific to each document type and code type to retrieve relevant coding guidance, historical examples, and compliance rules. Real-Time Processing Monitor 354 may present retrieval results to Users 102 and incorporate feedback to refine subsequent retrieval operations.

Knowledge bases may be stored locally, at the computing device implementing MAIA User Interface 300, or the knowledge bases may be stored by a networked system, networked with MAIA Outcome Feedback Computing System 100, such as MAIA Data Store 200, or knowledge bases may be part of a third-party system.

Knowledge bases are specific for each document type. For example, if the determined document type is an operative note document type, the uploaded medical claim data is routed to the Operative Note Document Pipeline 452 and a hybrid data retrieval process is implemented using the results of Operative Note Document Pipeline 452 on Operative Note Knowledge Base (“KB”) 262, using processes defined specifically for each knowledge base.

Operative Note KB 262 may include: CPT code definitions and descriptors; AAOS Musculoskeletal Coding Guide content; global period rules specifying pre-operative and post-operative periods by CPT code; add-on code parent-child relationships; NCCI bundling edits and modifier indicators; and CMS Relative Value Unit (RVU) data.

E&M KB 264 may include: MDM matrix definitions mapping component levels to E&M codes; two-of-three rule logic for MDM-based code selection; time-based E&M thresholds; new versus established patient criteria; and specialty-specific E&M coding guidance.

ICD-10 KB 266 may include: ICD-10-CM alphabetic index; ICD-10-CM tabular list with inclusion/exclusion notes; ICD-10-CM Official Guidelines for Coding and Reporting; SNOMED-CT to ICD-10-CM crosswalk mappings; and laterality/specificity requirements.

The HCPCS Knowledge Base 267 may include: the HCPCS national alphanumeric code set; descriptions for durable medical equipment (DME), drugs, and biologicals; National Coverage Determinations (NCD) and Local Coverage Determinations (LCD) for supply medical necessity; MUE (Medically Unlikely Edit) unit limits for supplies and injections; and payer-specific pricing or fee schedule data for non-physician services.

The CPT Knowledge Base 269 may include: CPT code definitions and full descriptors; AAOS Musculoskeletal Coding Guide content; global period rules specifying pre-operative and post-operative windows by code; add-on code parent-child relationships; NCCI bundling edits and modifier indicators; and CMS Relative Value Unit (RVU) data including Work, Practice Expense, and Malpractice components.

The uploaded medical claim data, and all of the results of the document processing pipeline and the results of the hybrid retrieval process may be input into an embedding model to generate and assign; at least one medical billing code, which may include, CPT, ICD-10 and HCPCS codes and modifiers, to the uploaded medical claim data, an initial confidence score to each of the at least one medical billing code, and a preliminary code justification summary for each of the at least one medical billing code, which may be based on the results of the embedding model. The embedding model may be implemented locally on the computing device implementing MAIA User Interface 300, or part of a networked system with the MAIA Outcome Feedback Computing System 100, such as MAIA Primary LLM Engine 600.

MAIA Primary LLM Engine 600 may notify Users 102 of the generated candidate codes and justifications via Real-Time Processing Monitor 354 and query for feedback, including code approval, code correction, modifier addition, or justification refinement. User feedback may be logged for model fine-tuning and accuracy improvement.

The initial confidence score for each candidate medical billing code is compared to Thresholds and Triggers 662 to determine an appropriate validation processing path. Thresholds and Triggers 662 may include: Confidence score thresholds (e.g., codes exceeding 0.85 confidence routed to Fast Path); Code complexity indicators (e.g., E&M codes near level boundaries routed to Complex Path); NCCI conflict flags indicating potential bundling issues; Multiple coding pathway indicators where more than one valid code assignment exists; Historical accuracy rates for specific code families; and Payer-specific denial risk indicators.

Based on threshold comparison, each candidate code is routed to Fast Path Validation 670 (approximately 90% of cases, approximately 2 seconds processing time) or Complex Path Validation 680 (approximately 10% of cases, approximately 12 seconds processing time).

Fast Path Validation 670 is implemented on candidate medical billing codes with initial confidence scores exceeding defined thresholds. Fast Path Validation 670 is optimized for high-throughput, low-latency processing of high-confidence cases, targeting sub-second validation times for the majority of code assignments.

Validation Methods: Fast Path Validation 670 executes deterministic validation rules, which may include one or more of: NCCI bundling edit verification to confirm code combinations are permitted, including Column 1/Column 2 edits and mutually exclusive code detection; Modifier requirement validation based on code relationships, anatomical considerations, and payer-specific rules, including distinct procedural service modifiers (-59, -XE, -XP, -XS, -XU), laterality modifiers (-LT, -RT, -50), and professional/technical component modifiers (-26, -TC); Global period validation to detect conflicts with prior procedures within pre-operative or post-operative windows, including same-day procedure conflict detection; MUE (Medically Unlikely Edit) unit limit verification and unit quantity logic validation; Add-on code parent verification to confirm add-on codes are paired with valid primary procedure codes; Laterality and anatomical consistency validation to ensure modifier usage matches documented anatomical sites; Age and gender appropriateness validation for demographic-restricted codes; Frequency and interval validation to enforce minimum time periods between repeated procedures; Duplicate service detection to identify same-code, same-date, same-provider conflicts; Diagnosis-to-procedure linkage validation to confirm medical necessity based on ICD-10 to CPT relationships; LCD/NCD (Local/National Coverage Determination) rule application for payer-specific medical necessity requirements; Place of service appropriateness validation including telehealth and ASC coverage rules; ICD-10 sequencing rule validation including primary diagnosis selection, manifestation/etiology ordering, and external cause code placement.

Complex Path Validation 680 is implemented on candidate medical billing codes with initial confidence scores below defined thresholds, complexity indicators suggesting coding ambiguity, or flags indicating multiple valid coding pathways. Complex Path Validation 680 may implement one or more of the following mechanisms:

Multi-Agent Consensus Architecture: Multi-Agent Manager 682 instantiates a plurality of agent personas, which may be implemented as: distinct large language models operating independently; a single model with varied prompting strategies; specialized expert models for different code families (e.g., E&M expert, surgical expert, diagnosis expert); or adversarial agent pairs comprising proposer and challenger agents. Each agent persona may be configured with distinct coding perspectives, such as conservative compliance-focused coding, revenue optimization within compliance boundaries, literal guideline interpretation, or payer-specific denial avoidance. Agent personas are assigned weighted scores based on historical accuracy, domain relevance, calibrated confidence, and organization-specific coding philosophy.

Reasoning and Inference Methods: Complex Path Validation 680 may implement advanced reasoning approaches, including: chain-of-thought reasoning wherein agents generate explicit step-by-step rationale before code assignment; tree-of-thought exploration wherein agents evaluate branching decision paths and select optimal coding pathways; self-consistency sampling wherein multiple independent reasoning paths are generated and aggregated; recursive refinement wherein initial code assignments are iteratively improved through self-critique; and contrastive reasoning wherein agents explicitly compare candidate codes and generate differential justifications.

Debate Orchestration: Debate Orchestrator 684 coordinates iterative evaluation rounds wherein each agent persona independently analyzes the clinical documentation with retrieved knowledge base context. Agents may propose, support, or challenge candidate medical billing codes.

MAIA Primary LLM Engine 600 may notify Users 102 of the generated candidate medical billing codes and justifications via Real-Time Processing Monitor 354 and query for feedback, including candidate code approval, code correction, modifier addition, or justification refinement. User feedback may be logged for model fine-tuning and accuracy improvement.

A final output is generated comprising the validated medical billing codes and the following components:

NCCI Compliance Validation, including: exhaustive pairwise code comparison (O(n²) for n codes); Column 1/Column 2 edit validation with modifier exception identification; modifier requirement verification; and Medically Unlikely Edit (MUE) unit limit enforcement.

RVU Sequencing and Payment Calculation, including: sorting codes by Relative Value Unit (RVU) to identify the primary procedure; applying multiple procedure payment reductions (MPPR) to secondary procedures; calculating base payment (Work RVU+Practice Expense RVU+Malpractice RVU)×Conversion Factor; applying modifier-based adjustments (e.g., -50 bilateral, -51 multiple procedure); applying Geographic Practice Cost Index (GPCI) adjustments; and generating a total expected payment estimate.

Justification Generation, including: per-code rationale citing specific documentation elements; evidence citations with character offsets or section references; guideline references from retrieved knowledge base content; and identification of the validation path (Fast Path or Complex Path) and confidence trajectory.

The final output is provided to Users 102 via MAIA User Interface 300.

The final output may comprise validated or consensus medical billing codes including, CPT Codes, Modifiers, E/M levels, ICD-10 Codes, HCPCS Codes, descriptions, modifiers, confidence scores, RVU values, payment estimates, coding justifications, metadata, and validation and processing methods.

MAIA Outcome Feedback Computing System 100 may implement an end-to-end revenue cycle management platform by: generating validated medical billing codes from clinical documentation; formatting and submitting claims to third-party payers via EDI 837 transactions or payer portals; tracking claim status through adjudication; and executing Outcome Feeback System 800 in response to claim denials. Denial outcomes and payer feedback are used for accuracy tuning, threshold adjustment, and model fine-tuning to continuously improve coding accuracy.

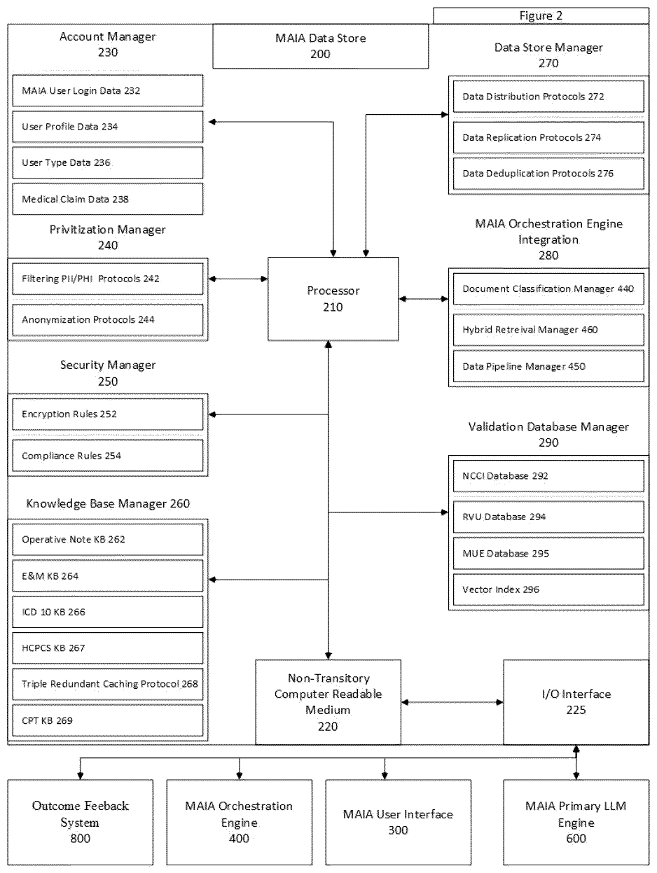

FIG. 2 illustrates MAIA Data Store 200. Data Store Manager 270 may operate MAIA Data Store 200 as a single data store or as a distributed data store utilizing Data Distribution Protocols 272. MAIA Data Store 200 comprises at least one processor 210 coupled to at least one non-transitory computer-readable medium 220. MAIA Data Store 200 may include a plurality of input/output (I/O) devices such as; keyboards, mouse or mice, displays, touch screen displays, voice command interfaces, optical readers, scanners, cameras, microphones and wired and wireless network devices, connected via I/O Interface 225.

Data Distribution Protocols 272 may include horizontal partitioning to balance workloads across storage nodes, vertical partitioning to optimize query performance by co-locating frequently accessed columns, and tenant-based partitioning to isolate data by healthcare organization for compliance and performance.

MAIA Data Store 200 may store structured, semi-structured, and unstructured data utilizing a plurality of storage methods, including: relational databases for transactional data, audit logs, and structured coding results; NoSQL databases for flexible document storage and high-write-throughput logging; vector databases for embedding storage and similarity search; in-memory databases for low-latency caching and session state; and object storage for clinical document archives.

MAIA Data Store 200 may implement logical separation between protected health information (PHI) storage and de-identified analytics data, enabling compliant data processing workflows. Users 102 may define data format, storage methods, and distribution protocols, or these may be automatically configured by Data Store Manager 270 based on data classification and compliance requirements.

Data Store Manager 270 may implement Data Replication Protocols 274, including: full replication for complete data redundancy across geographic regions; incremental replication for efficient synchronization of changed data; snapshot replication for point-in-time recovery and audit purposes; and streaming replication for real-time failover capability.

Replication strategies may be configured to meet healthcare compliance requirements, including geographic data residency restrictions and disaster recovery time objectives. Users 102 may define replication techniques and performance thresholds, or these may be automatically configured by Data Store Manager 270 based on data criticality classification.

Data Replication Protocols 274 may implement audit-preserving replication wherein all data modifications are replicated with complete audit metadata including timestamp, user identifier, source system, and modification type. Audit-preserving replication ensures that replicated data maintains full chain-of-custody documentation required for healthcare compliance audits and legal discovery, with cryptographic integrity verification across replica sets.

Data Store Manager 270 may utilize Data Deduplication Protocols 276 to optimize storage, including: in-line deduplication for real-time duplicate detection during data ingestion; post-process deduplication for batch optimization of stored data; whole-file deduplication for identifying duplicate clinical documents; block-level deduplication for optimizing storage of similar documents with shared content; and sub-file deduplication for identifying duplicate sections within clinical notes (e.g., templated text, standard headers, copied-forward content).

Users 102 may authenticate to MAIA Outcome Feedback Computing System 100 via Account Manager 230, which validates login credentials against MAIA User Login Data 232. User accounts include User Profile Data 234 containing user-specific preferences and configuration, and User Type Data 236 defining role-based access permissions. User Type Data 236 may include medical coders, billing specialists, physicians, practice administrators, compliance auditors, and payer representatives.

If the matched user account has upload permission, which may be defined in User Profile Data 234 or the permission may be automatic as part of specific user types, for example, if the matched user account is a medical provider user type, or a third-party insurance provider user type, said user types may automatically have upload permission, permitted Users 102 may upload medical claim data via Medical Claim Upload Manager 350 which allows Users 102 to upload medical claim data in a plurality of formats, including PDF Upload 351, Text Upload 352, OCR 353, Real-Time Processing Monitor 354, EHR Integration 355, Voice to Text 356 and A.I. Scribe 357.

The uploaded clinical documentation may include protected health information (PHI) and personally identifiable information (PII). Prior to active storage, Privatization Manager 240 may process the uploaded data using:

Filtering PHI/PII Protocols 242 to detect and remove or tokenize patient identifiers including names, dates, medical record numbers, social security numbers, and other HIPAA-defined identifiers; and

Anonymization Protocols 244 to apply statistical anonymization techniques ensuring re-identification risk falls below defined thresholds.

Security Manager 250 enforces Compliance Rules 254 governing data handling, use, storage, and transmission. Clinical documentation may be stored and transmitted in encrypted format as determined by Encryption Rules 252, which may specify encryption algorithms, key management protocols, and encryption-at-rest and encryption-in-transit requirements.

Compliance Rules 254 and Encryption Rules 252 may be system-defined based on regulatory requirements (e.g., HIPAA, HITECH, state privacy laws), organization-defined based on internal policy, or payer-defined based on contractual requirements.

MAIA Data Store 200 may implement Encounter Boundary Isolation, wherein retrieved patient historical data is stored with explicit temporal and encounter boundary metadata distinguishing it from current encounter data. Encounter Boundary Isolation prevents inappropriate bleeding of historical clinical indicators (e.g., prior surgical complications, historical risk factors) into current encounter coding assessments, while preserving access to historical context for legitimate coding purposes such as global period validation and chronic condition continuity. Document Processing Pipeline Manager 450 may query historical data with explicit boundary constraints to ensure extraction agents operate on appropriately scoped clinical content.

After the uploaded medical claim data has been modified by Privatization Manager 240 and Security Manager 250, document classification is then performed on the medical claim data, to determine a document type, and the medical claim data is intelligently routed to at least one document processing pipeline based on the determined document type. The document classification, intelligent routing, and data processing pipeline may be performed locally, on the computing device implementing MAIA Data Store 200, utilizing MAIA Orchestration Engine Integration 280, or as part of a networked system, networked with MAIA Outcome Feedback Computing System 100, such as MAIA Orchestration Engine 400, implementing Document Classification Manager 440 and Document Processing Pipeline Manager 450.

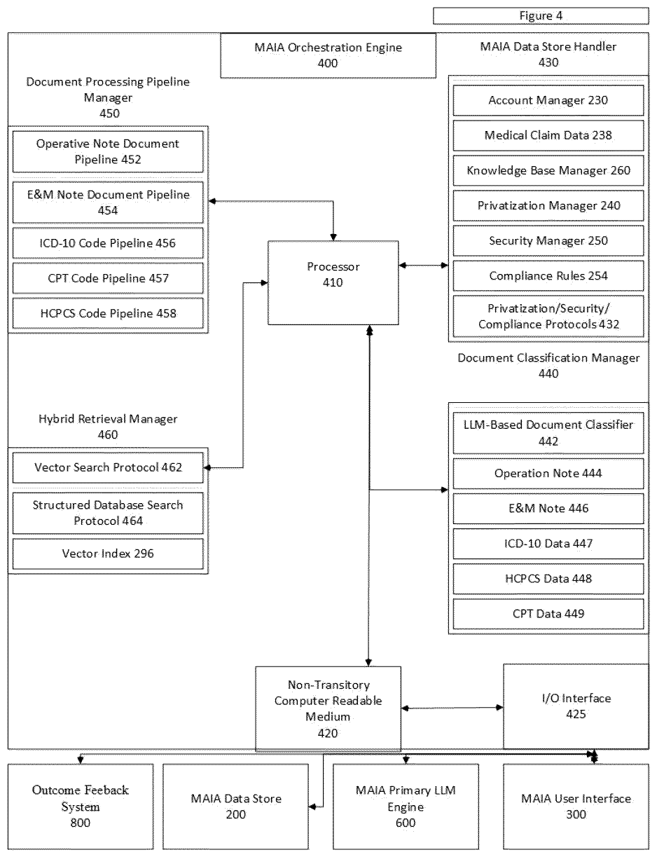

Each document processing pipeline implements data transformations, analytics, and feature extraction as described in relation to FIG. 4. Hybrid Retrieval Manager 460 performs vectorized and structured database searches on document-type-specific knowledge bases, including Operative Note KB 262, E&M KB 264, ICD-10 KB 266, HCPCS KB 267 and CPT KB 269.

Knowledge Base Manager 260 manages knowledge base storage, indexing, and retrieval. Knowledge Base Manager 260 may implement Triple Redundant Caching Protocol 268, comprising: a first cache tier for frequently accessed rules and code definitions stored in in-memory cache; a second cache tier for moderately accessed content stored in fast SSD-backed cache; and a third cache tier for less frequently accessed content stored in distributed cache with geographic replication. Cache invalidation is coordinated across tiers when knowledge base content is updated.

Knowledge Base Manager 260 may implement Knowledge Base Version Control, wherein all knowledge base updates (e.g., annual CPT updates, quarterly NCCI updates, LCD revisions) are versioned with effective dates. When processing clinical documentation, MAIA Outcome Feedback Computing System 100 may retrieve knowledge base content as of the date of service, enabling accurate coding based on rules in effect at the time of the encounter rather than current rules. This supports retrospective coding, audit defense, and appeals where historical rule interpretation is relevant.

Knowledge Base Version Control maintains full history of rule changes with provenance metadata identifying the source (CMS, AMA, payer) and effective date range for each rule version.

MAIA Data Store 200 may store extraction provenance metadata for each processed document, including: model versions used for extraction; prompt templates applied; retrieved knowledge base content with version identifiers; and intermediate extraction outputs at each pipeline stage. Extraction provenance enables reproducible coding, wherein a historical coding decision can be exactly replicated by restoring the system state (model versions, knowledge base versions, configuration) that produced the original result. This supports audit defense, appeals, and debugging of coding errors.

Hybrid Retrieval Manager 460 may implement Retrieval Fusion protocols to combine results from Vector Search Protocol 462 and Structured Database Search Protocol 464. Fusion methods may include:

Reciprocal Rank Fusion (RRF), wherein results from each retrieval method are ranked and combined using reciprocal rank weighting;

Score-based fusion, wherein normalized similarity scores from each method are combined with configurable weights;

Cascade fusion, wherein structured search results are used to filter or re-rank vector search results; and

Query-dependent fusion, wherein fusion weights are dynamically adjusted based on query characteristics (e.g., specific code lookups weight structured search; ambiguous clinical descriptions weight vector search).

Medical Claim Data 238, extracted features, and retrieved knowledge base data are input into MAIA Primary LLM Engine 600 for medical billing code generation. MAIA Primary LLM Engine 600 implements large language model inference to analyze clinical documentation in context with retrieved coding guidance and generates:

At least one candidate medical billing code, which may include CPT procedure codes, ICD-10-CM diagnosis codes, HCPCS codes, and associated modifiers;

An initial confidence score for each candidate code representing the model's assessment of code accuracy; and

A preliminary code justification summary citing specific documentation elements and guideline references supporting each code assignment.

Code generation may be performed locally on the computing device implementing MAIA User Interface 300 or remotely via MAIA Primary LLM Engine 600. Generated codes, confidence scores, and justifications are stored in MAIA Data Store 200 and associated with Medical Claim Data 238.

MAIA Primary LLM Engine 600 may generate decomposed confidence scores comprising multiple sub-scores, including:

Documentation sufficiency confidence (does the documentation contain required elements?);

Code specificity confidence (is this the most specific applicable code?);

Medical necessity confidence (does the diagnosis support the procedure?);

Compliance confidence (does the code pass known validation rules?); and

Historical accuracy confidence (how accurate has the model been on similar cases?).

Decomposed confidence enables targeted intervention when specific confidence components are low while others are high, rather than treating confidence as a single opaque score.

For each candidate medical billing code, the initial confidence score is compared to Thresholds and Triggers 662 to determine an appropriate validation processing path. Validation paths vary in computational complexity, accuracy optimization, and processing time requirements.

Codes with confidence scores exceeding defined thresholds are routed to Fast Path Validation 670 for rapid deterministic validation. Codes with confidence scores below defined thresholds, or with complexity indicators suggesting coding ambiguity, are routed to Complex Path Validation 680 for multi-agent consensus validation.

Validation path outputs, including validated codes, updated confidence scores, and validation summaries, are stored in MAIA Data Store 200 and associated with Medical Claim Data 238.

Thresholds and Triggers 662 may implement Adaptive Threshold Learning, wherein confidence thresholds are dynamically adjusted based on observed outcomes. For code families or payers with elevated denial rates despite high initial confidence, thresholds may be automatically tightened to route more cases to Complex Path Validation. For code families with consistently accurate Fast Path results, thresholds may be relaxed to improve throughput. Threshold adjustments may be constrained by configurable bounds to prevent runaway adaptation.

Validation path outputs are transmitted to Validation Database Manager 290 for final compliance validation. Validation Database Manager 290 performs:

NCCI Validation using NCCI Database 292, which stores National Correct Coding Initiative edit pairs including Column 1/Column 2 edits, mutually exclusive codes, and modifier indicators. NCCI Database 292 is updated quarterly per CMS publication schedule.

RVU Sequencing Validation using RVU Database 294, which stores Relative Value Unit data including Work RVU, Practice Expense RVU (facility and non-facility), Malpractice RVU, conversion factors, and GPCI adjustment factors. RVU Database 294 is updated annually per CMS Physician Fee Schedule.

Validation Database Manager 290 generates final output including validated codes, compliance check results, RVU-sequenced procedure order, and payment estimates. Final output is stored in MAIA Data Store 200 and associated with Medical Claim Data 238.

Validation Database Manager 290 may implement MUE Database 295 storing Medically Unlikely Edit unit limits by CPT code. MUE validation may be enhanced with claim history context, wherein unit limits are evaluated not just against the current claim but against cumulative units submitted for the same patient, same code, and same date of service across multiple claims. This prevents MUE violations that would only become apparent when multiple claims are aggregated by the payer.

Medical Claim Data 238 with validated billing codes may be submitted automatically to insurance providers based on configurable submission rules, including confidence thresholds, validation status, and organization-specific approval workflows.

Claim status is tracked through adjudication, including submission confirmation, pending status, payment posting, and denial notification.

In the event of a denial, the original Medical Claim Data 238, validated codes, and denial information are transmitted to Outcome Feeback System 800 for appeal or correction processing. Denial outcomes are used for accuracy tuning, feeding back into model fine-tuning, threshold adjustment, and validation rule enhancement.

Prior to claim submission, MAIA Outcome Feedback Computing System 100 may implement Pre-Submission Denial Prediction, wherein a machine learning model estimates the probability of denial for each claim based on: Code combinations and modifiers; Payer and plan type; Diagnosis-procedure relationships; Historical denial patterns for similar claims; Documentation completeness indicators; and Provider-specific denial history with the target payer.

Claims with elevated denial risk may be flagged for additional review, documentation enhancement, or alternative coding pathways before submission, reducing denial rates and rework.

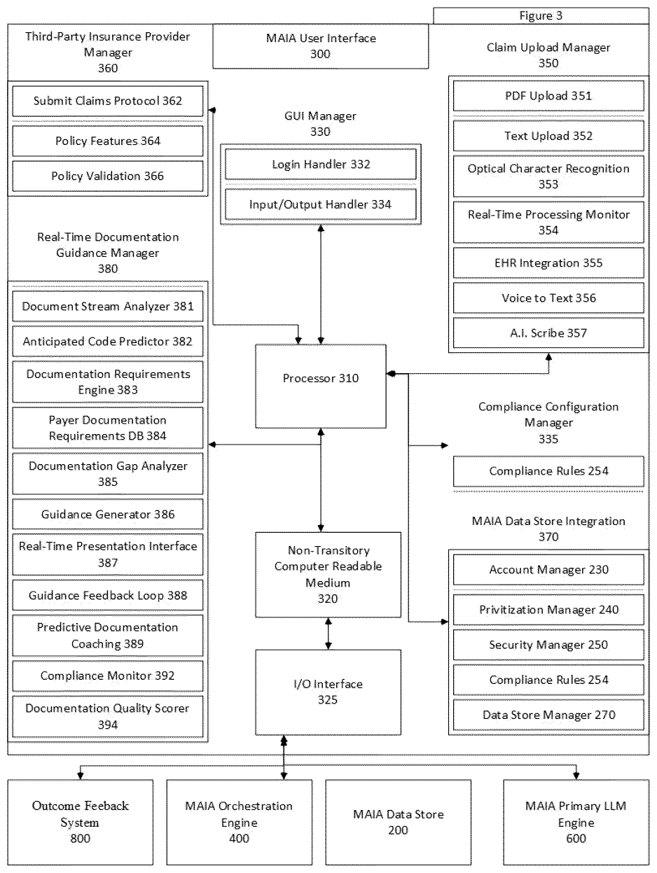

FIG. 3 illustrates MAIA User Interface 300. MAIA User Interface 300 may include a GUI displayed on at least one display of a computing device comprising at least one processor 310 coupled to at least one non-transitory computer readable medium 320. The Displayed GUI may be executed by GUI Manager 330 and allows a user to access Account Manager 230 and input login information to access their MAIA user account via Login Handler 332, and Input/Output Handler 334 via I/O Interface 325. MAIA User Interface 300 may include a plurality of input/output (I/O) devices such as; keyboards, mouse or mice, displays, touch screen displays, voice command interfaces, optical readers, scanners, cameras, microphones, wired and wireless network devices connected via I/O Interface 325. MAIA User Interface 300 may integrate with MAIA Data Store 200 via MAIA Data Store Integration 370.

User authentication is performed as described in relation to FIG. 1. Account Manager 230 validates login credentials against MAIA User Login Data 232 using authentication methods including username/password, multi-factor authentication, single sign-on (SSO), and biometric authentication.

User accounts include User Profile Data 234 containing user-specific configuration and preferences, and User Type Data 236 defining role-based permissions. User types may include medical coders, billing specialists, physicians, practice administrators, compliance officers, and payer representatives.

Permitted Users 102 may upload clinical documentation via Medical Claim Upload Manager 350, which supports multiple input formats and methods: PDF Upload 351 for scanned or electronic PDF documents; Text Upload 352 for plain text clinical notes; OCR 353 for image-based document conversion; EHR Integration 355 for direct extraction from electronic health record systems via HL7 FHIR, HL7 v2, or CCD/C-CDA interfaces; Voice to Text 356 for direct clinician dictation transcription; and A.I. Scribe 357 for ambient clinical documentation capture, wherein patient-clinician encounters are recorded and automatically structured into clinical note format. Real-Time Processing Monitor 354 provides status updates and user feedback throughout upload and processing workflows.

Medical Claim Upload Manager 350 may implement Intelligent Format Detection, wherein uploaded documents are automatically analyzed to determine format type, structure, and content organization without requiring user specification. Format detection may identify: Document type (operative report, progress note, consultation, lab results); EHR source system based on formatting patterns and templates; Section structure and header conventions; and Encoding, character set, and embedded content (images, tables).

Following format detection, Document Normalization converts diverse input formats into a standardized internal representation optimized for downstream processing pipelines. Clinical documentation may be uploaded in structured or unstructured format and stored in pre-defined or user-defined data structures. Processing modes include: Individual processing for single-document, interactive coding with immediate feedback; Batch processing for high-volume offline processing with aggregated results; and Real-time processing with continuous user feedback via Real-Time Processing Monitor 354, enabling intervention at each processing stage.

Clinical documentation may include patient data containing protected health information (PHI) and personally identifiable information (PII), which is handled in accordance with Compliance Rules 254.

If User Type Data 236 indicates a System Administrator or Compliance Officer user type, MAIA User Interface 300 may implement Compliance Configuration Manager 335. Compliance Configuration Manager 335 allows authorized Users 102 to add, remove, and modify Compliance Rules 254 governing data handling, privacy protocols, retention policies, and coding policies within MAIA Outcome Feedback Computing System 100. All compliance rule modifications are logged with user attribution, timestamp, and change description for audit purposes. Compliance Configuration Manager 335 may implement Compliance Rule Impact Preview, wherein proposed rule changes are simulated against recent claim data before deployment. Impact Preview generates reports including: Number of claims that would be affected; Coding changes that would result from the new rule; Potential revenue impact (positive or negative); Compliance risk indicators; and Conflicts with existing rules. Impact Preview enables safe iteration on compliance rules without affecting production operations.

Patient data within Medical Claim Data 238 and retrieved historical data may be de-identified, anonymized, or tokenized locally at MAIA User Interface 300 or remotely via Privatization Manager 240, in accordance with Security Manager 250 and Compliance Rules 254.

Real-Time Processing Monitor 354 may query Users 102 for de-identification preferences, including: Level of de-identification (full removal, tokenization with reversible linkage, or anonymization); Data elements to preserve for coding purposes (dates, ages, anatomical locations); and Organization-specific privacy policy selection.

Privatization Manager 240 may implement Contextual De-identification, wherein de-identification methods are adapted based on downstream coding requirements. For example: Patient age may be preserved (or binned to age ranges) when age-specific codes apply; Dates may be shifted consistently rather than removed when global period calculations require temporal relationships; Anatomical location identifiers may be preserved when laterality coding is required; and Provider identifiers may be tokenized rather than removed when incident-to billing determination is needed. Contextual De-identification balances privacy protection with coding accuracy requirements. Hybrid retrieval is performed as described in relation to FIG. 1 and FIG. 4.

Real-Time Processing Monitor 354 may present retrieval results to Users 102, including: Retrieved coding guidelines and rules; Similar historical cases with known codes; Relevant policy citations; and Retrieval confidence scores.

Users 102 may provide feedback including relevance ratings, additional search terms, and manual retrieval of supplementary content. User feedback may be used to refine retrieval rankings and improve retrieval model performance.

Real-Time Processing Monitor 354 may implement Retrieval Explanation, wherein each retrieved knowledge base entry includes an explanation of why it was retrieved, including: Matching terms or concepts between the clinical documentation and retrieved content; Embedding similarity score and nearest-neighbor rank; Structured query matches (code lookups, rule triggers); and Retrieval path (vector search, structured search, or both). Retrieval Explanation enables Users 102 to understand and validate retrieval results, building trust in system recommendations and enabling targeted feedback.

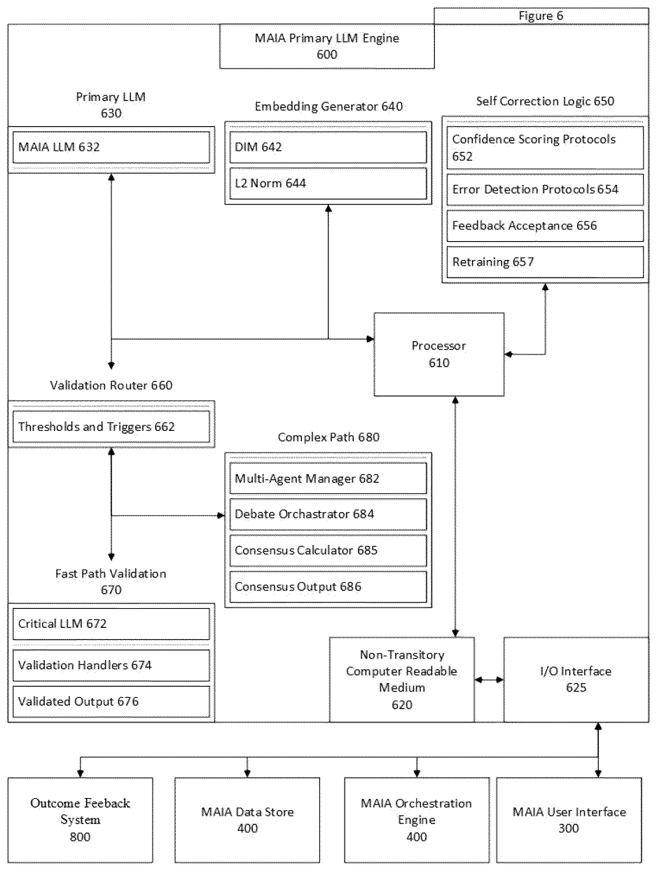

Medical billing code generation is performed by MAIA Primary LLM Engine 600 as described in relation to FIG. 1 and FIG. 6. Real-Time Processing Monitor 354 may present generated codes, confidence scores, and justifications to Users 102 and solicit feedback, including: Code approval or correction; Modifier additions or changes; Justification enhancement or correction; and Documentation sufficiency flags.

Based on user feedback, MAIA Primary LLM Engine 600 may regenerate codes with modified prompts incorporating user input, retrieved additional context, or adjusted reasoning parameters. Iterative generation continues until user approval or maximum iterations. User feedback is logged and may be used for model fine-tuning, prompt optimization, and retrieval improvement.

MAIA Primary LLM Engine 600 may implement Feedback-Guided Iterative Refinement, wherein user feedback on generated codes triggers targeted regeneration. Rather than regenerating from scratch, the system: Identifies the specific aspect of the output that user feedback addressed (code selection, modifier, specificity); Retrieves additional context relevant to the identified issue; Regenerates with a focused prompt emphasizing the corrected aspect; and Presents revised output with explanation of changes. Feedback-Guided Iterative Refinement reduces user effort by converging quickly on correct codes rather than requiring complete manual correction.

The initial confidence score for each of the at least one medical billing codes is compared to a set of Thresholds and Triggers 662 to determine one, from a plurality of validation processing paths, of varying complexity and processing requirements, such as Fast Path Validation 670, or Complex Path Validation 680 to perform on each of the at least one medical billing codes. Users 102 may be presented with the selected validation path for each of the at least one medical billing codes and asked to submit feedback or corrections via Real-Time Processing Monitor 354.

MAIA User Interface 300 may implement User-Defined Validation Policies, wherein Users 102 or organization administrators configure validation routing rules based on organizational risk tolerance. Policies may specify: Code families requiring mandatory Complex Path validation regardless of confidence; Payers requiring enhanced validation due to historical audit activity; Dollar thresholds above which Complex Path is mandatory; New coder oversight rules requiring validation review for training periods; and Compliance-critical code categories with zero-tolerance for Fast Path. Validation policies enable organizations to balance throughput against risk based on their specific requirements.

A final output is generated comprising the output of the selected validation path, validation checks, payment calculations and a final justification comprising code rationale and the validation procedures for each of the validated medical billing codes. Users 102 may be presented with the final output for each of validated medical billing codes and asked to submit feedback or corrections via Real-Time Processing Monitor 354.

Prior to final approval, MAIA User Interface 300 may display a Pre-Submission Quality Score representing the overall confidence that the claim will be paid without denial or audit. Quality Score may be computed from: Code confidence scores; Validation pass/fail results; Documentation completeness assessment; Payer-specific denial risk prediction; Historical accuracy on similar cases; and Compliance risk indicators. Quality Score enables Users 102 to prioritize review effort on lower-quality claims and confidently release high-quality claims with minimal review.

Each of the successfully validated, at least one medical billing codes, may be submitted to Third-Party Insurance Provider Manager 360, which may proceed with a Submit Claims Protocol 362 to submit each of the validated at least one medical billing codes to third-party insurance providers. Medical billing codes submitted to third-party insurance providers may be tracked and monitored, and in the event of a denial of coverage, Medical Claim Data 238, the final output, and the denial are sent to Outcome Feeback System 800, which automatically generates a denial response.

If User Type Data 236 indicates a Third-Party Payer user type, Users 102 may access Third-Party Insurance Manager 360 to submit payer-specific policy content via Policy Features 364, including: Medical necessity criteria defining coverage requirements for specific procedures; Prior authorization requirements identifying procedures requiring pre-approval; Modifier rules specifying payer-specific modifier requirements beyond NCCI; Bundling edits defining payer-specific code bundling policies; Documentation requirements specifying required documentation elements for specific codes; Preferred coding guidance indicating payer preferences for ambiguous coding scenarios; and Fee schedule data for accurate payment estimation. Payer-submitted policy content is incorporated into payer-specific knowledge bases and used to train payer-aware models, enabling coding optimized for each payer's requirements. Third-Party Insurance Manager 360 may implement Policy Validation 366, wherein payer-submitted policies are validated for consistency and conflicts before incorporation. Validation checks may include: Conflict detection between payer policy and CMS national rules; Syntax and format validation for structured policy rules; Historical consistency checking against prior payer submissions; and Impact simulation showing how the policy would affect coding for sample claims. Policy conflicts are flagged for resolution, and policy history is maintained for audit and dispute purposes.

MAIA Outcome Feedback Computing System 100 may include Real-Time Documentation Guidance Manager 380, which provides concurrent feedback and suggestions to physicians and clinical staff as they author clinical documentation. Real-Time Documentation Guidance Manager 380 analyzes documentation in progress, anticipates likely procedure and diagnosis codes, identifies documentation gaps that may impact coding accuracy or payer acceptance, and provides actionable guidance to strengthen documentation before note finalization. By intervening at the point of documentation rather than after claim submission or denial, Real-Time Documentation Guidance Manager 380 addresses documentation deficiencies proactively, reducing downstream coding errors, claim denials, and revenue leakage.

Documentation Stream Analyzer 381 monitors clinical documentation as it is authored, receiving real-time input from EHR systems via integration APIs, ambient clinical documentation systems, voice-to-text transcription systems, or direct text input interfaces.

Documentation Stream Analyzer 381 performs continuous analysis of in-progress documentation including procedure identification, wherein references to procedures, surgeries, or clinical interventions are identified as they are documented. Documentation Stream Analyzer 381 also performs diagnosis extraction, wherein diagnostic terms, clinical findings, and impression language are extracted and mapped to potential ICD-10-CM codes. Documentation Stream Analyzer 381 also performs service context detection, wherein the clinical setting, patient type (new versus established), and service category (evaluation and management, surgical, diagnostic) are determined.

Documentation Stream Analyzer 381 also performs payer context integration, wherein the patient's insurance coverage is retrieved to enable payer-specific guidance. Documentation Stream Analyzer 381 also performs historical context retrieval, wherein prior visit documentation, problem lists, and treatment history are retrieved to inform documentation requirements.

Documentation Stream Analyzer 381 operates with minimal latency to provide guidance while the clinician is actively documenting, rather than after documentation is complete.

Anticipated Code Predictor 382 predicts the medical billing codes likely to result from the documentation in progress based on partial documentation analysis. For procedure code prediction, Anticipated Code Predictor 382 analyzes procedure descriptions, anatomical references, and surgical approach language to predict CPT and HCPCS codes. For example, documentation mentioning “arthroscopic” and “knee” and “meniscectomy” predicts CPT codes in the 29880-29881 range.

For evaluation and management prediction, Anticipated Code Predictor 382 analyzes the emerging MDM (Medical Decision Making) elements including problem complexity indicators, data review references, and risk management language to predict the likely E&M code level.

For diagnosis code prediction, Anticipated Code Predictor 382 maps diagnostic language to ICD-10-CM codes, identifying specificity requirements such as laterality, acuity, and anatomical detail that affect code selection.

Anticipated Code Predictor 382 updates predictions continuously as documentation progresses, refining code predictions as additional detail is added.

Documentation Requirements Engine 383 determines the documentation elements required to support the anticipated codes and satisfy payer requirements.

Code-Specific Requirements include the documentation elements required by CPT, ICD-10, and E&M coding guidelines to support code assignment. For example, E&M level 99215 requires documentation of moderate or high complexity MDM with specific element thresholds. Surgical codes require documentation of the specific procedure performed, anatomical site, and surgical approach.

Payer-Specific Requirements include documentation elements required by the patient's specific payer beyond standard coding requirements. Requirements vary by payer and plan and may include medical necessity language demonstrating why the service was required, failed conservative treatment documentation showing that less invasive approaches were attempted, prior authorization reference numbers, specific clinical criteria defined in Local Coverage Determinations (LCDs) or payer medical policies, and attestation language required by certain payers.

Documentation Requirements Engine 383 accesses Payer Documentation Requirements Database 384, which stores payer-specific documentation requirements organized by payer, plan, procedure code, and diagnosis code. Payer Documentation Requirements Database 384 may be populated via payer policy extraction from published coverage policies and medical necessity criteria, LCD and NCD analysis from Medicare coverage determinations, denial pattern inference from documentation elements correlated with claim denials, clearinghouse data integration from aggregated payer requirement data, and manual entry from staff-entered requirements based on payer communications.

Payer Documentation Requirements Database 384 may be stored locally, at the computing device implementing MAIA User Interface 300, or the Payer Documentation Requirements Database 384 may be stored by a networked system, networked with MAIA Outcome Feedback Computing System 100, such as MAIA Data Store 200, or Payer Documentation Requirements Database 384 may be part of a third-party system.

Documentation Gap Analyzer 385 compares the documentation in progress against the requirements identified by Documentation Requirements Engine 383 to identify gaps. Documentation Gap Analyzer 385 identifies missing elements, which are required documentation components that have not yet been documented. Documentation Gap Analyzer 385 also identifies insufficient specificity, which occurs when documentation is present but lacks required detail such as laterality not specified or acuity not indicated.

Documentation Gap Analyzer 385 also identifies unsupported assertions, which occur when conclusions or diagnoses are stated without supporting clinical evidence documented. Documentation Gap Analyzer 385 also identifies payer-specific gaps, which are elements required by the specific payer that are not present in documentation.

Guidance Generator 386 transforms identified gaps into actionable, clinician-friendly guidance presented in real-time.

Guidance types include element prompts, which are suggestions to add specific documentation elements such as “Consider documenting laterality (left/right) for the meniscal tear.”

Guidance types also include template suggestions, which are pre-written phrases or templates the clinician can insert such as “Insert: Patient has failed six weeks of conservative treatment including physical therapy and NSAIDs.”

Guidance types also include payer alerts, which are notifications of payer-specific requirements such as “Aetna requires explicit documentation of failed conservative treatment for arthroscopic knee procedures.”

Guidance types also include code impact warnings, which are alerts when documentation gaps may result in lower code levels or claim denials such as “Current documentation supports 99214; adding complexity elements could support 99215.”

Guidance types also include medical necessity prompts, which are suggestions for medical necessity language such as “Document the clinical indication for MRI: what symptoms or findings necessitate this imaging?”

Guidance is prioritized by impact, with high-revenue-impact and high-denial-risk gaps surfaced first.

Real-Time Presentation Interface 387 delivers guidance to clinicians through integration with clinical documentation workflows. EHR Integration presents guidance within the electronic health record interface, appearing as sidebar suggestions, inline prompts, or notification alerts as the clinician documents. The integration is designed to be minimally intrusive while ensuring guidance is visible.

Ambient Scribe Integration presents guidance within ambient clinical documentation interfaces, where AI scribes transcribe patient encounters. Guidance may be presented to the clinician during or after the encounter, or incorporated into AI scribe drafts for clinician review.

Voice Interface Integration presents guidance via audio prompts for clinicians using voice-to-text documentation, allowing hands-free notification of documentation needs.

Mobile Interface presents guidance via mobile device for clinicians who document or review notes on tablets or smartphones.

Guidance Presentation Modes include passive mode, wherein guidance is displayed but clinician action is not required; active mode, wherein guidance requires clinician acknowledgment or response; and smart mode, wherein presentation intensity adapts based on gap severity, clinician preferences, and historical response patterns.

Clinicians may accept guidance by incorporating suggested elements, dismiss guidance with optional reason capture, or defer guidance for later review.

Documentation Guidance Feedback Loop 388 captures clinician responses to guidance and downstream outcomes to continuously improve guidance relevance and effectiveness.

Guidance acceptance tracking monitors which guidance suggestions clinicians accept, modify, or dismiss, identifying guidance types that are helpful versus those that are ignored or dismissed.

Outcome correlation tracks the relationship between documentation guidance acceptance and downstream outcomes including coding accuracy, claim acceptance, and denial rates. Guidance that correlates with improved outcomes is reinforced; guidance with no outcome impact may be deprioritized.

Clinician preference learning adapts guidance presentation to individual clinician preferences, reducing guidance for documentation elements a clinician consistently includes without prompting, and emphasizing guidance for elements the clinician tends to omit.

Payer requirement updates incorporate new payer requirements discovered through denial analysis into Payer Documentation Requirements Database 384 and future guidance.

False positive reduction identifies guidance that is frequently dismissed as unnecessary and adjusts thresholds to reduce low-value interruptions.

Real-Time Documentation Guidance Manager 380 may implement Specialty-Specific Guidance Modules tailored to the documentation patterns and coding requirements of specific medical specialties.

Orthopedic Surgery Module provides guidance specific to musculoskeletal procedures including anatomical specificity requirements (joint, bone, muscle identification), laterality documentation, surgical approach documentation (arthroscopic, open, percutaneous), implant and device documentation for HCPCS coding, and AAOS coding guideline alignment.

Evaluation and Management Module provides guidance specific to E&M encounters including MDM element documentation for problem complexity, data complexity, and risk, time-based documentation requirements, new versus established patient indicators, and prolonged services documentation thresholds.

Diagnostic Imaging Module provides guidance for imaging orders and interpretations including clinical indication documentation for medical necessity, comparison study references, findings specificity, and laterality and anatomical detail.

Additional specialty modules may be implemented for cardiology, gastroenterology, neurology, and other specialties with distinct documentation and coding requirements.

Real-Time Documentation Guidance Manager 380 may implement Predictive Documentation Coaching 389, wherein documentation needs are anticipated before the clinical encounter based on scheduled services and patient context.

Pre-encounter briefing provides clinicians with documentation reminders before scheduled procedures or visits based on the scheduled service type, the patient's payer and known payer requirements, the patient's history and anticipated documentation needs, and common documentation gaps for similar encounters.

Encounter type templates suggest documentation templates optimized for the specific encounter type, pre-populated with required elements and prompts for variable content.

Similar case analysis retrieves documentation from similar prior cases that resulted in successful claim payment, providing examples of documentation that satisfied payer requirements.

Real-Time Documentation Guidance Manager 380 may implement Compliance Monitor 392, which identifies documentation patterns that may create compliance or audit risk.

Upcoding risk detection identifies documentation that may not support the anticipated code level, warning clinicians when documentation appears insufficient for high-level codes.

Cloning detection identifies documentation that appears to be copied from prior notes without appropriate modification, which may trigger audit scrutiny.

Medical necessity alignment verifies that documented diagnoses support the medical necessity of documented procedures, flagging misalignments that may result in medical necessity denials.

Audit target identification flags documentation patterns associated with increased audit risk based on OIG (Office of Inspector General) work plans, RAC (Recovery Audit Contractor) targets, and payer audit patterns.

Real-Time Documentation Guidance Manager 380 may implement Documentation Quality Scorer 394, which provides clinicians with a real-time quality score for documentation in progress.

Quality dimensions include completeness (are all required elements present), specificity (is sufficient detail provided), medical necessity support (does documentation support the clinical need for services), payer alignment (does documentation satisfy known payer requirements), and coding supportability (will documentation support accurate code assignment).

Quality score visualization presents the quality score graphically within the documentation interface, allowing clinicians to see documentation strength improve as they add elements.

Threshold alerts notify clinicians when documentation quality falls below acceptable thresholds, prompting additional documentation before note finalization.

Quality benchmarking compares documentation quality against peer benchmarks, departmental standards, and historical personal performance.

FIG. 4 illustrates MAIA Orchestration Engine 400. Users 102 input login data to MAIA User Interface 300, once the login data is matched to a MAIA Outcome Feedback Computing System 100 user account, if the matched user has upload permission, Users 102 may upload Medical Claim Data 238. Medical Claim Data 238. Historical patient data may be retrieved using Medical Claim Data 238. MAIA Orchestration Engine 400 comprises at least one processor 410 coupled to at least one non-transitory computer-readable medium 420. MAIA Orchestration Engine 400 may include a plurality of input/output (I/O) devices such as; keyboards, mouse or mice, displays, touch screen displays, voice command interfaces, optical readers, scanners, cameras, microphones and wired and wireless network devices, connected via I/O Interface 425.

Clinical documentation may be de-identified prior to transmission to MAIA Orchestration Engine 400, or MAIA Orchestration Engine 400 may invoke Privatization Manager 240 and Security Manager 250 via MAIA Data Store Handler 430 using Privatization/Security/Compliance Protocols 432. This architectural flexibility enables deployment configurations where PHI never leaves the local environment, or where centralized de-identification is preferred for consistency and auditability.

Document Classification Manager 440 performs document classification on uploaded clinical documentation. Document Classification Manager 440 may implement: Rule-based classification using keyword patterns, section headers, and document structure; Machine learning classification using trained models on document features; and LLM-based classification using large language model inference to analyze document content and LLM-Based Document Classifier 442.

Document types include Operative Note 444 (surgical/procedural documentation), E&M Note 446 (evaluation and management encounters), and ICD-10 Data 447 (diagnosis-focused documentation), HCPCS Data 448 (implant and device documentation), and CPT Data 449 (procedural documentation).

Based on classification results, Document Processing Pipeline Manager 450 routes clinical documentation to one or more processing pipelines, including Operative Note Document Pipeline 452, E&M Note Document Pipeline 454, and ICD-10 Code Pipeline 456. Documents may be routed to multiple pipelines simultaneously. Document Processing Pipeline Manager 450 performs document-type-specific data transformations and feature extraction. Extraction methods for each document type may be implemented as: Deterministic rules for structured extraction of well-defined elements; Machine learning models for pattern-based extraction; and LLM-based extraction for complex semantic interpretation. Extraction rules and policies may be user-defined, system-wide, or governed by Compliance Rules 254.

For example, Operative Note Document Pipeline 452 performs feature extraction on operative reports and surgical documentation, including: Primary and secondary procedure identification with procedure descriptions; Anatomical site extraction with laterality indicators (left, right, bilateral); Surgical approach identification (open, arthroscopic, percutaneous, endoscopic, laparoscopic); Distinct procedural step enumeration for component coding; Implant, device, and graft identification for supply coding; Anesthesia type and time documentation;

Surgical findings and complexity indicators; and Time documentation for time-based codes. Extracted features are used by Hybrid Retrieval Manager 460 to perform retrieval-augmented generation against Operative Note KB 262. Final code validation includes add-on code protection, NCCI bundling validation, and RVU-based procedure sequencing.

E&M Note Document Pipeline 454 performs feature extraction on evaluation and management documentation using specialized extraction agents:

Encounter Boundary Detection Agent isolates current encounter documentation from historical patient data, preventing inappropriate inclusion of prior visit indicators in current encounter coding.

Problem Complexity Agent extracts and categorizes diagnoses from Assessment/Impression sections, determining problem status (new, existing, acute, chronic, worsening) and counting unique addressable problems.

Data Complexity Agent extracts reviewed and ordered data from HPI, Plan, and Results sections, categorizing by data type (labs, imaging, external records, independent interpretation) and source (internal, external).

Risk Complexity Agent analyzes the Plan section to identify current management decisions, including prescription drug management, minor and major procedures ordered, and hospitalization decisions.

Time Agent extracts total time documentation and time-based activity descriptions when time-based E&M coding is applicable.

Combiner Agent applies the two-of-three rule to extracted MDM component levels (problem complexity, data complexity, risk complexity) to determine the appropriate E&M code level.

Metadata Filtering Agent isolates relevant note sections for each extraction agent based on document structure and section header recognition.

ICD-10 Code Pipeline 456 performs feature extraction for diagnosis coding, including: Diagnostic term extraction from Assessment, Impression, and Diagnosis sections; Clinical indicator identification including signs, symptoms, and abnormal findings; Laterality and anatomical specificity extraction; Acuity indicators (acute, chronic, recurrent); Causal relationship identification for manifestation and etiology coding; External cause indicators (injury mechanism, place of occurrence); and Complication and comorbidity identification. Extracted diagnostic features are mapped to candidate ICD-10-CM codes using medical ontology mapping (e.g., SNOMED-CT to ICD-10-CM crosswalks), alphabetic index term matching, and tabular list navigation against ICD-10 KB 266.

Hybrid Retrieval Manager 460 performs hybrid data retrieval using extracted features and clinical documentation content. The hybrid retrieval process combines: Vector Search Protocol 462 using Vector Index 296 for semantic similarity search; and Structured Database Search Protocol 464 for exact-match rule and code lookups. Retrieval is performed against document-type-specific knowledge bases to gather relevant coding guidance, rules, and examples.

Knowledge base content and search protocols are specific to each document type: Operative Note KB 262 contains CPT procedure code definitions, AAOS Musculoskeletal Coding Guide content, global period rules, add-on code relationships, NCCI bundling edits, and RVU data. Retrieval queries include procedure-to-code mapping, add-on code eligibility, and bundling edit lookup. E&M KB 264 contains MDM matrix definitions, two-of-three rule logic, time-based E&M thresholds, new/established patient criteria, and prolonged services rules. Retrieval queries include MDM level determination and E&M code selection. ICD-10 KB 266 contains alphabetic index terms, tabular list entries with inclusion/exclusion notes, Official Coding Guidelines, and SNOMED-CT crosswalks. Retrieval queries include diagnostic term-to-code mapping and specificity requirements.

Medical Claim Data 238, document classification results, extracted features from document processing pipelines, and retrieved knowledge base content from Hybrid Retrieval Manager 460 are input into MAIA Primary LLM Engine 600 for medical billing code generation. MAIA Primary LLM Engine 600 implements large language model inference to analyze clinical documentation in context with retrieved coding guidance. For each clinical document, MAIA Primary LLM Engine 600 generates: At least one candidate medical billing code, which may include CPT procedure codes, ICD-10-CM diagnosis codes, HCPCS codes, and associated modifiers; An initial confidence score for each candidate code representing model certainty; and A preliminary code justification summary citing specific documentation elements and guideline references supporting the code assignment.

For each candidate medical billing code, Validation Router 660 compares the initial confidence score to Thresholds and Triggers 662 to determine an appropriate validation processing path. Thresholds and Triggers 662 may include: Confidence score thresholds (e.g., codes above 0.90 confidence routed to Fast Path); Code complexity indicators (e.g., E&M codes near level boundaries routed to Complex Path); Code value thresholds (e.g., high-RVU codes requiring enhanced validation); NCCI conflict flags indicating potential bundling issues; Historical accuracy indicators for specific code families; and Payer-specific risk flags based on denial history. Based on threshold comparison, each code is routed to Fast Path Validation 670 or Complex Path Validation 680, which vary in computational complexity, accuracy optimization, and processing time.

Validation Router 660 may implement Dynamic Threshold Adjustment, wherein confidence thresholds are temporarily adjusted based on system workload and processing queue depth. During high-volume periods, thresholds may be tightened to route more cases to Fast Path, maintaining throughput. During low-volume periods, thresholds may be relaxed to route more cases to Complex Path for enhanced accuracy. Dynamic adjustment balances accuracy and throughput based on real-time operational conditions.

A final output is generated for each validated medical billing code, comprising: Validated codes from the selected validation path (Fast Path Validated Output 676 or Consensus Output 686); Compliance validation results including NCCI edit checks, modifier validation, and MUE verification; RVU sequencing with primary procedure designation and multiple procedure reductions; Payment calculation including base RVU, conversion factor, modifier adjustments, and geographic adjustments; Total expected payment estimate; and Final justification comprising code rationale, documentation citations, guideline references, and validation path identification.

MAIA Primary LLM Engine 600 may implement Justification Quality Scoring, wherein generated code justifications are evaluated for completeness and audit defensibility. Quality scoring may assess: Presence of required documentation elements for the assigned code level; Specificity of citation (character-level vs. section-level references); Guideline alignment (explicit mapping to published coding guidelines); Absence of contradictory documentation; and Consistency with historical justifications for similar codes. Justifications with low quality scores may trigger additional documentation retrieval or coder review before finalization.

Successfully validated medical billing codes are submitted to payers via Third-Party Insurance Provider Manager 360, which implements Submit Claims Protocol 362. Third-Party Insurance Provider Manager 360 supports multiple submission channels: EDI 837 professional and institutional claim transactions; Clearinghouse integration for multi-payer routing; Direct payer portal submission via API or RPA; and Paper claim generation for payers requiring physical submission.

Third-Party Insurance Provider Manager 360 tracks claim status through adjudication, including submission acknowledgment, pending status, payment posting, and denial notification. Upon denial, Medical Claim Data 238, final output, and denial information are transmitted to Outcome Feedback System 800 for automated correction or appeal processing.

Third-Party Insurance Provider Manager 360 may implement Intelligent Submission Routing, wherein the optimal submission channel is selected for each claim based on: Payer preferences and acceptance rates by channel; Claim complexity (simple claims via automated EDI; complex claims via portal with attachments); Historical processing speed by channel; Attachment requirements (some payers require clinical documentation upload); and Cost optimization (EDI typically lower cost than portal or clearinghouse). Intelligent routing maximizes clean claim acceptance rates and minimizes submission costs.

MAIA Orchestration Engine 400 may implement Pipeline Execution Optimization, wherein document processing pipelines are executed in parallel when documents are routed to multiple pipelines, and pipeline stages are pipelined to overlap extraction and retrieval operations. Optimization strategies may include: Parallel pipeline execution across available compute resources; Speculative pipeline execution when classification confidence is moderate (execute likely pipelines before classification finalizes); Result caching for common extraction patterns; and Early termination when high-confidence codes are identified before all pipelines complete.

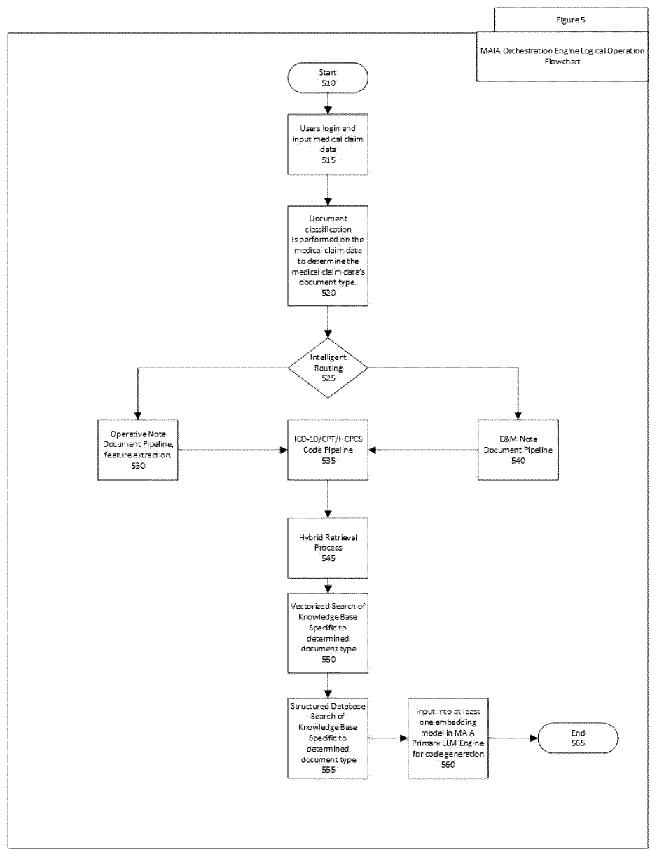

FIG. 5 illustrates a logical operation flowchart of MAIA Orchestration Engine 400.
Step 510: Process Start.
Step 515: Users 102 authenticate and upload clinical documentation (Medical Claim Data 238).
Step 520: Document Classification Manager 440 determines document type.
Step 525: Clinical documentation is routed to at least one document pipeline based on document type.
Step 530: Operative Note Document Pipeline 452 performs feature extraction (if applicable).
Step 535: ICD-10 Code Pipeline 456 performs feature extraction (if applicable).
Step 540: E&M Note Document Pipeline 454 performs feature extraction (if applicable).
Step 545: Hybrid retrieval is initiated using extracted features.
Step 550: Vector search queries document-type-specific knowledge bases using semantic similarity.
Step 555: Structured database search queries document-type-specific knowledge bases using exact-match rules.
Step 560: Clinical documentation, extracted features, and retrieved knowledge base content are transmitted to MAIA Primary LLM Engine 600 for code generation.
Step 565: Orchestration process Ends; processing continues at MAIA Primary LLM Engine 600.

FIG. 6 illustrates MAIA Primary LLM Engine 600, comprising at least one processor 610 coupled to at least one non-transitory computer-readable medium 620. Processor 610 may include: Graphics processing units (GPUs) optimized for parallel tensor operations; Tensor processing units (TPUs) optimized for neural network inference; Application-specific integrated circuits (ASICs) for efficient LLM inference; or General-purpose CPUs with vector extensions for smaller models. MAIA Primary LLM Engine 600 may include a plurality of input/output (I/O) devices such as; keyboards, mouse or mice, displays, touch screen displays, voice command interfaces, optical readers, scanners, cameras, microphones, wired and wireless network devices connected via I/O Interface 625.

MAIA Primary LLM Engine 600 may be deployed as a standalone inference service, integrated within MAIA Orchestration Engine 400, or distributed across multiple inference nodes with load balancing for scalability. MAIA Primary LLM Engine 600 may implement Heterogeneous Model Deployment, wherein different model sizes and architectures are deployed on different hardware tiers: Large models (highest accuracy) deployed on high-memory GPU nodes for complex cases; Medium models (balanced accuracy/speed) deployed on standard GPU nodes for typical cases; Small/distilled models (fastest inference) deployed on CPU or edge nodes for simple cases; and Quantized models deployed on cost-optimized infrastructure for batch processing.

Validation Router 660 routes cases to appropriate model tiers based on complexity indicators, enabling cost-performance optimization. Document classification is performed by Primary LLM 630 using MAIA LLM 632 to determine document type. MAIA LLM 632 may implement one or more large language models, including: General-purpose large language models with broad language understanding; Medical domain-specific large language models pre-trained on clinical literature and documentation; and Lightweight classification models optimized for fast document type determination.

Document types include Operative Note 444, E&M Note 446, ICD-10 Data 447, HCPCS Data 448 and CPT Data 449.

Based on classification results, clinical documentation is routed to Operative Note Document Pipeline 452, E&M Note Document Pipeline 454, and/or ICD-10 Code Pipeline 456. Documents may be routed to multiple pipelines simultaneously. Document Classification Manager 440 may implement Cascaded Classification, wherein a fast, lightweight classifier first attempts classification, and cases where the lightweight classifier has low confidence are escalated to a more capable (but slower) LLM classifier. Cascaded classification reduces average latency by handling easy cases quickly while maintaining accuracy on difficult cases.

Document Processing Pipeline Manager 450 performs document-type-specific data transformations and feature extraction on clinical documentation. Each pipeline extracts features optimized for its code type: Operative Note Document Pipeline 452 extracts procedure descriptions, anatomical sites, laterality, surgical approach, and procedural components; E&M Note Document Pipeline 454 extracts MDM elements via specialized agents (Problem, Data, Risk, Time) with encounter boundary isolation; and ICD-10 Code Pipeline 456 extracts diagnostic terms, clinical indicators, laterality, and acuity markers.

Hybrid Retrieval Manager 460 performs hybrid data retrieval using extracted features and clinical documentation content. The hybrid process combines: Vector Search Protocol 462 using Vector Index 296 to perform semantic similarity search, identifying knowledge base entries with high embedding similarity to clinical content; and Structured Database Search Protocol 464 to perform exact-match queries for specific codes, modifier rules, bundling edits, and coverage policies. Retrieval is performed against document-type-specific knowledge bases (Operative Note KB 262, E&M KB 264, ICD-10 KB 266, HCPCS KB 267 and CPT KB 269) to gather relevant coding guidance.

Hybrid Retrieval Manager 460 may implement Context-Aware Retrieval Depth, wherein the number of retrieved knowledge base entries is dynamically adjusted based on: Document complexity (more retrieval for complex multi-procedure cases); Initial classification confidence (more retrieval when document type is uncertain); Code specificity requirements (more retrieval for codes requiring detailed guideline interpretation); and Historical retrieval-to-accuracy correlation (retrieving more when additional context has historically improved accuracy). Dynamic retrieval depth optimizes the trade-off between context completeness and prompt length limits. MAIA Primary LLM Engine 600 receives as input: clinical documentation; extracted features; and retrieved knowledge base content.

Embedding Generator 640 may generate vector embeddings of clinical documentation for use in similarity search and retrieval. Embedding representations may vary in dimensionality (DIM) 642 and similarity may be computed using distance metrics such as L2 (Euclidean) norm 644 or cosine similarity.

Primary LLM 630 performs medical billing code generation, producing: At least one candidate medical billing code; An initial confidence score for each candidate code; and A preliminary code justification summary.

Self Correction Logic 650 performs automatic error detection and confidence calibration. Confidence Scoring Protocols 652 calibrate raw model confidence based on historical accuracy for similar cases. Error Detection Protocols 654 identify potential coding errors, including: Codes inconsistent with extracted anatomical indicators; Codes outside typical range for the identified specialty; Code combinations flagged by preliminary compliance screening; and Confidence patterns indicating model uncertainty.

Self Correction Logic 650 may implement Self-Consistency Verification, wherein the LLM is prompted multiple times with slightly varied prompts or temperature settings, and the consistency of generated codes across runs is measured. Codes that are consistently generated across multiple runs are assigned higher confidence; codes that vary across runs indicate uncertainty and may be routed to Complex Path Validation or human review. Self-consistency provides an additional uncertainty signal beyond single-pass confidence scores.

Validation Router 660 compares initial confidence scores to Thresholds and Triggers 662 to route each candidate code to an appropriate validation path: Codes with confidence scores exceeding defined thresholds and no complexity flags are routed to Fast Path Validation 670; Codes with confidence scores below thresholds, complexity indicators, or risk flags are routed to Complex Path Validation 680. Routing decisions may also consider code value (RVU), payer-specific denial history, and audit risk indicators.

Fast Path Validation 670 is implemented on candidate codes with confidence scores exceeding defined thresholds. Fast Path Validation 670 executes: Critical LLM 672, a lightweight language model optimized for fast validation inference, which performs secondary review of code-documentation alignment; Validation Handlers 674, deterministic rule executors that apply compliance checks including NCCI edit verification, modifier validation, global period checking, and MUE enforcement. Critical LLM 672 and Validation Handlers 674 may operate in series (sequential validation) or in parallel (concurrent validation with result aggregation). Fast Path Validation 670 generates Fast Path Validated Output 676, comprising:

Validated medical billing codes; Updated confidence scores incorporating validation results; Validation rule results summary; and Processing time and resource metrics. Validation models may be trained or fine-tuned using payer-specific policy features and historical accuracy data via Feeback Acceptance 656 and Retraining 657.

Fast Path Validation 670 may implement Validation Rule Learning, wherein new validation rules are automatically inferred from patterns in historical denials and corrections. When denials cluster around specific code combinations, modifier patterns, or documentation characteristics, the system proposes candidate validation rules for review. Approved rules are added to Validation Handlers 674, continuously expanding the rule base without manual rule authoring.

Complex Path Validation 680 is implemented on candidate codes with confidence scores below thresholds or with complexity indicators. Complex Path Validation 680 implements a multi-agent consensus mechanism:

Multi-Agent Manager 682 instantiates a plurality of agent personas, each configured with distinct coding perspectives: Conservative Compliance Agent prioritizing audit defensibility and conservative code selection; Revenue Optimization Agent identifying supported higher-value codes within compliance boundaries; Guideline-Strict Agent applying literal interpretation of coding rules; Payer-Aware Agent incorporating historical denial patterns for the target payer; and Specialty Expert Agent applying specialty-specific coding conventions. Each persona is assigned a weighted score based on historical accuracy, domain relevance, and organization-specific coding philosophy.

Debate Orchestrator 684 coordinates iterative evaluation rounds wherein each agent persona independently analyzes the clinical documentation and proposes, supports, or challenges candidate codes with cited rationale. Debate continues until convergence or maximum rounds.

Consensus Calculator 685 aggregates weighted agent votes to compute a consensus score for each candidate code. Consensus methods may include weighted voting, Bayesian aggregation, or game-theoretic mechanisms. Once consensus threshold is reached, Consensus Output 686 is generated, comprising: Consensus medical billing codes; Final confidence scores; Consensus justification with per-agent rationale; and Debate summary highlighting key disagreements and resolution.

Complex Path Validation 680 may implement Disagreement-Triggered Escalation, wherein cases with persistent agent disagreement (consensus not reached within defined rounds) are automatically escalated to human coder review. The escalation package includes: All agent proposals with supporting rationale; Points of disagreement and debate highlights; Relevant retrieved guidelines and similar historical cases; and Recommended focus areas for human review.

Human decisions on escalated cases are logged and used to calibrate agent weights and improve future consensus. A final output is generated comprising validated codes from the selected validation path and the following components:

NCCI Compliance Validation: Exhaustive pairwise code comparison (O(n²) for n codes); Column 1/Column 2 edit validation with modifier exception identification; Modifier requirement verification; Medically Unlikely Edit (MUE) unit limit enforcement; and Mutually exclusive code detection.

RVU Sequencing and Payment Calculation: Sorting codes by Relative Value Unit (RVU) to identify primary procedure; Applying multiple procedure payment reductions (MPPR) to secondary procedures; Base payment calculation (Work RVU+PE RVU+MP RVU)×Conversion Factor; Modifier-based adjustments (bilateral, assistant surgeon, multiple procedure); Geographic Practice Cost Index (GPCI) adjustments; and Total expected payment estimate.

Justification Generation: Per-code rationale citing specific documentation; Evidence citations with section/character references; Guideline references from retrieved knowledge base; and Validation path identification (Fast Path or Complex Path).

Final output is provided to Users 102 via MAIA User Interface 300. Final output generation may include Payment Variance Analysis, wherein the expected payment is compared to: Historical average payment for the same code combination; Benchmark payment for the same codes at peer organizations; Expected payment under alternative coding approaches. Significant variances trigger alerts for review, helping identify potential under-coding, over-coding, or payer-specific payment anomalies.

The final output may comprise: Validated CPT procedure codes with modifiers; ICD-10-CM diagnosis codes with sequencing; HCPCS supply, drug, and equipment codes; Code descriptions; Final confidence scores; RVU values (Work, PE, MP); Expected payment estimates; Per-code justifications with documentation citations; Processing metadata (timestamps, model versions, pipeline paths); and Audit trail data (validation path, rule results, agent votes if applicable). Output format and included fields may be user-configured or automatically determined based on submission requirements.

Each of the successfully validated, at least one medical billing codes, may be submitted to Third-Party Insurance Provider Manager 360, which may proceed with a Submit Claims Protocol 362 to submit each of the validated at least one medical billing codes to third-party insurance providers. Medical billing codes submitted to third-party insurance providers may be tracked and monitored, and in the event of a denial of coverage, Medical Claim Data 238, the final output, and the denial are sent to Outcome Feeback System 800, which automatically generates a denial response.

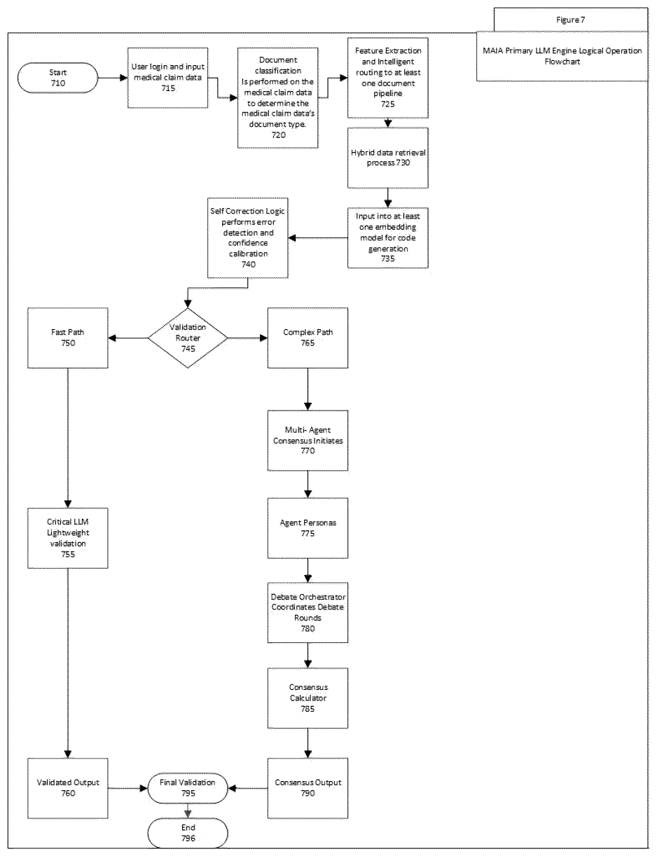

FIG. 7 illustrates a logical operation flowchart of MAIA Primary LLM Engine 600. Step 710: Process Start.
Step 715: Clinical documentation received (authentication and upload performed per FIG. 1). User login and input medical claim data.
Step 720: Document classification determines document type.
Step 725: Document processing pipelines perform feature extraction.
Step 730: Hybrid data retrieval process gathers knowledge base content via vector and structured search.
Step 735: Clinical documentation, features, and retrieved content input to Primary LLM 630 for code generation, which may include at least one embedding model.
Step 740: Self Correction Logic 650 performs error detection and confidence calibration.
Step 745: Validation Router 660 routes codes to validation paths based on confidence.
Step 750: Fast Path branch (codes exceeding confidence threshold).
Step 755: Critical LLM 672 performs lightweight validation.
Step 760: Fast Path Validated Output 676 generated.
Step 765: Complex Path branch (codes below confidence threshold).
Step 770: Multi-Agent Manager 682 initiates consensus process.
Step 775: Agent Personas instantiated with distinct perspectives.
Step 780: Debate Orchestrator 684 coordinates evaluation rounds.
Step 785: Consensus Calculator 685 aggregates results.
Step 790: Consensus Output 686 generated.
Step 795: Final validation checks (NCCI, RVU sequencing, payment calculation) performed on Validated or Consensus Output.
Step 796: Process ends; final output provided to Users 102.

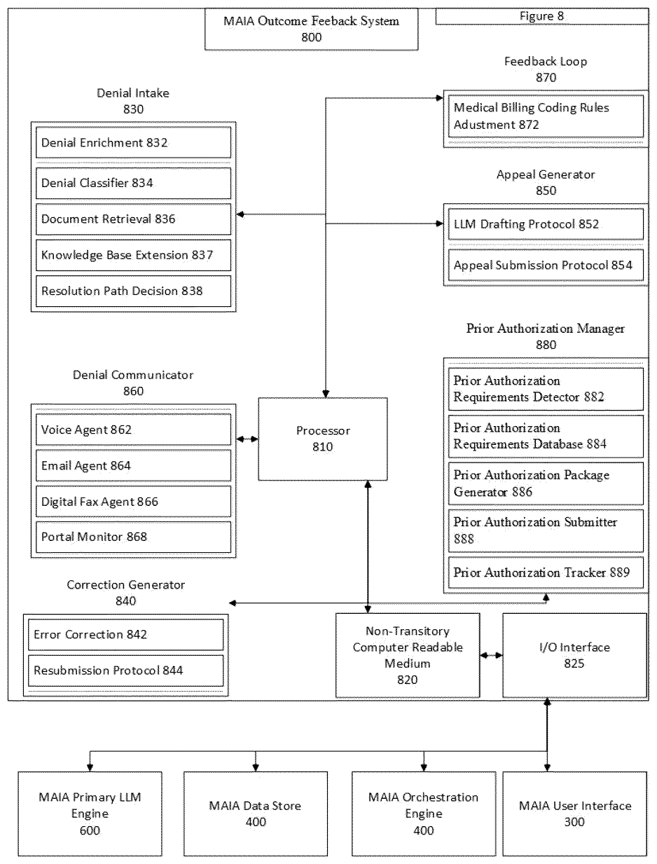

FIG. 8 illustrates Outcome Feeback System 800, comprising at least one processor 810, at least one non-transitory computer-readable medium 820, and I/O Interface 825 for communication with external systems. Outcome Feedback System 800 may include a plurality of input/output (I/O) devices such as; keyboards, mouse or mice, displays, touch screen displays, voice command interfaces, optical readers, scanners, cameras, microphones, wired and wireless network devices connected via I/O Interface 825.

Upon receipt of a claim denial, Denial Intake 830 ingests denial information from multiple channels: EDI 835 remittance transactions with CARC/RARC denial reason codes;

Payer portal denial notifications and EOB documents; Electronic remittance advice (ERA) files; and Scanned paper EOBs processed via OCR.

Denial Enrichment 832 extracts and enriches denial information: Parsing denial reason codes and remark codes; Extracting denied CPT/ICD codes and line items; Linking denial to original Medical Claim Data 238 and source clinical documentation; Retrieving original code justifications and validation results; and Gathering historical denial patterns for the same payer/code combination.

Denial Enrichment 832 may implement Denial Similarity Clustering, wherein incoming denials are compared to historical denials using embedding similarity to identify clusters of similar denial patterns. Clustering enables: Batch resolution of similar denials with common correction or appeal strategies; Root cause identification when denial clusters correlate with specific coding patterns; Prioritization of denial investigation when new clusters emerge; and

Template reuse for appeal generation when similar appeals have succeeded historically.

Denial Classifier 834 categorizes denials into types based on reason codes and enrichment data: Eligibility denials: patient not covered, coverage terminated, coordination of benefits issues; Authorization denials: prior authorization required/expired/mismatch; Coding/billing denials: invalid code, unbundling edit, modifier error, duplicate claim; Medical necessity denials: LCD/NCD criteria not met, diagnosis doesn't support procedure; Global/bundled denials: service in global period, service bundled with another code; Administrative denials: timely filing, missing information, claim format errors. Each denial type is associated with designated resolution pathways optimized for that category.

Document Retrieval 836 automatically gathers appeal packet components: Clinical documentation from source EHR (operative reports, progress notes, diagnostic results);

Prior authorization records and approval documentation; Payer-required forms (appeal forms, medical necessity forms); Supporting evidence (lab results, imaging reports, pathology);

Original claim submission and remittance history; and Prior appeal correspondence if applicable.

Document Retrieval 836 may implement Intelligent Document Assembly, wherein appeal packet contents are dynamically selected based on denial type and payer requirements: Medical necessity denials receive clinical evidence prioritized by relevance to coverage criteria; Coding denials receive targeted documentation supporting the disputed code; Authorization denials receive authorization correspondence and timeline documentation. Document assembly optimizes appeal packet size and relevance while ensuring completeness.

Knowledge Base Extension 837 retrieves payer-specific content relevant to the denial: Payer medical policies from policy databases, payer portals, and structured feeds; LCD/NCD (Local/National Coverage Determination) policies for Medicare; Payer-specific global period rules and bundling edits; Payer-specific modifier requirements and documentation standards; and

Historical appeal outcomes for similar denials with the target payer.

Resolution Path Decision 838 determines the optimal resolution strategy based on denial type, root cause analysis, and historical success rates: Correction and resubmission for correctable errors (demographics, modifiers, coding errors); or Formal appeal for coverage disputes, medical necessity challenges, or policy interpretation disagreements.

Resolution Path Decision 838 may implement Cost-Benefit Resolution Analysis, wherein the expected value of each resolution path is computed: Expected Value=(Probability of Success×Recovery Amount)-(Resolution Cost) Factors include: Historical success rates for similar denials/resolutions; Denied amount and recovery potential; Resource cost (automated vs. human effort, time to resolution); Opportunity cost of pursuing low-probability appeals. Resolution paths are recommended based on expected value optimization, enabling efficient allocation of denial management resources.

If correction is selected, Correction Generator 840 generates corrected claim data. Error Correction 842 applies appropriate corrections based on denial reason: Demographic corrections (patient/provider identifiers, policy numbers); CPT/ICD code corrections or substitutions; Modifier additions, removals, or corrections; Unit quantity adjustments; Place of service corrections; Referring/ordering provider additions; Timely filing indicator updates; and Frequency code corrections for recurring services.

Resubmission Protocol 844 submits corrected claims with appropriate corrected claim indicators (e.g., frequency code 7 for replacement) and original claim references. Correction Generator 840 may implement Correction Validation, wherein proposed corrections are validated against the same compliance rules used in original coding before resubmission: NCCI edit verification on corrected code combinations; Modifier appropriateness validation; Documentation sufficiency confirmation for new codes; Payer-specific rule checking. Correction Validation prevents resubmission of claims that will fail for different reasons, reducing rework cycles.

If appeal is selected, Appeal Generator 850 generates an appeal packet. LLM Drafting Protocol 852 uses large language model inference to generate a structured appeal letter comprising: Patient/Claim Identification: patient demographics, claim number, date of service, denied codes; Reconsideration Request: clear statement requesting payment reconsideration with specific relief requested; Clinical Summary: relevant clinical findings extracted from medical records, with citations to operative notes, progress notes, and diagnostic results; Policy Citation: applicable coverage policy (LCD, NCD, payer medical policy) with specific criteria sections; Evidence Mapping: explicit point-by-point mapping of documentation elements to policy criteria, demonstrating that coverage requirements are satisfied; and Conclusion: summary of argument and requested action. Generated appeals may be reviewed by Users 102 before submission or submitted automatically based on confidence thresholds via Appeal Submission Protocol 854.

Denial Communicator 860 manages multi-channel communication for denial resolution:

Voice Agent 862 conducts automated or agent-assisted phone interactions with payer representatives for status inquiries, expedited review requests, peer-to-peer scheduling, and information gathering. Voice Agent 862 may implement speech recognition, natural language understanding, and text-to-speech for automated interactions.

Email Agent 864 monitors incoming email for payer correspondence, parses email content to extract status updates and information requests, and generates follow-up communications.

Digital Fax Agent 866 monitors incoming faxes for payer correspondence, parses fax content to extract status updates and information requests, and generates follow-up communications.

Portal Monitor 868 accesses payer portals via API integration or robotic process automation (RPA) to retrieve appeal status, download determination letters, submit documentation, and track processing milestones. Status updates from all channels are aggregated and associated with the denial record in MAIA Data Store 200.

Denial Communicator 860 may implement Proactive Follow-Up Scheduling, wherein follow-up actions are automatically scheduled based on: Payer-specific expected response times;

Regulatory timelines (e.g., state prompt-pay laws, Medicare appeal deadlines); Escalation triggers when expected response times are exceeded; Optimal follow-up timing patterns learned from historical data. Proactive scheduling ensures denials don't age out while awaiting response, and optimizes follow-up timing for maximum effectiveness.

Feedback Loop 870 implements closed-loop learning from denial outcomes to continuously improve coding accuracy. Medical Billing Code Rules Adjustment 872 may apply: Threshold adjustments: tightening confidence thresholds for code families with elevated denial rates; Validation rule enhancement: adding new validation rules based on observed denial patterns; Knowledge base updates: incorporating denial rationale and successful appeal arguments into knowledge base content; Model fine-tuning signals: flagging denial cases as training examples for model improvement; Retrieval optimization: adjusting retrieval weights to prioritize content that would have prevented denials; Alert generation: notifying coding managers of emerging denial patterns requiring attention; and Agent persona calibration: adjusting multi-agent weights based on which perspectives would have predicted denials.

Feedback Loop 870 may implement Causal Denial Attribution, wherein each denial is traced back through the coding pipeline to identify the root cause: Documentation insufficiency: clinical note did not contain required elements; Extraction failure: system failed to extract relevant information; Retrieval gap: relevant guideline not retrieved during hybrid retrieval;

Model error: LLM generated incorrect code despite adequate context; Validation miss: validation rules failed to catch the error; External cause: payer error, coverage change, patient eligibility issue. Causal attribution enables targeted improvement—documentation issues feedback to extraction tuning, model errors to fine-tuning, validation misses to rule enhancement.

MAIA Outcome Feedback Computing System 100 may include Prior Authorization Manager 880, which automates the identification, generation, and submission of prior authorization requests required by third-party payers before rendering certain medical services. Prior Authorization Manager 880 operates in coordination with MAIA Orchestration Engine 400 to intercept clinical documentation at or before the point of service, determine prior authorization requirements, and initiate authorization workflows when required.

Prior Authorization Requirements Detector 882 analyzes extracted diagnosis codes (ICD-10-CM) and procedure codes (CPT, HCPCS) in conjunction with patient coverage information to determine whether prior authorization is required before service rendering or claim submission.

Determining prior authorization requirements is complex because requirements vary at multiple levels of specificity. Payer-level variation exists because different payers maintain different prior authorization requirements based on their coverage policies. Plan-level variation exists because within a single payer, different plan types (HMO, PPO, high-deductible, Medicare Advantage, Medicaid managed care) may have different authorization requirements for the same procedure. Code-level variation exists because authorization requirements are specified at the procedure code level, with some codes always requiring authorization, some never requiring authorization, and some requiring authorization only in combination with certain diagnoses or patient characteristics. Temporal variation exists because authorization requirements change over time as payers update their policies, often with limited advance notice.

Prior Authorization Requirements Detector 882 accesses Prior Authorization Requirements Database 884, which stores authorization requirements organized by payer, plan, and procedure code. Prior Authorization Requirements Database 884 may be populated via clearinghouse integration, wherein integration with healthcare clearinghouses such as Availity, Waystar, or similar services that maintain aggregated prior authorization requirements across multiple payers enables real-time eligibility and authorization requirement lookups for specific patient, plan, and procedure combinations. Prior Authorization Requirements Database 884 may also be populated via payer API integration, wherein direct integration with payer prior authorization APIs where available, including HIPAA-compliant X12 278 Health Care Services Review transactions, provides real-time authorization determination. Prior Authorization Requirements Database 884 may also be populated via payer portal data extraction, wherein automated or semi-automated extraction of authorization requirements from payer portal documentation and published coverage policies maintains current requirement data. Prior Authorization Requirements Database 884 may also be populated via contract data ingestion, wherein structured import of authorization requirements from payer contracts and provider manuals provides contractually-specified requirements.

Prior Authorization Requirements Database 884 may also be populated via manual maintenance, wherein staff-entered authorization requirements based on payer communications, denial patterns, and institutional knowledge captures requirements not available through automated channels. Prior Authorization Requirements Database 884 may also be populated via inference from denial history, wherein machine learning inference of unstated authorization requirements based on patterns of authorization-related denials identifies requirements not explicitly documented by payers.

Prior Authorization Requirements Detector 882 queries Prior Authorization Requirements Database 884 using the patient's verified payer and plan information from practice management system integration, combined with the extracted procedure and diagnosis codes, to determine whether prior authorization is required.

When Prior Authorization Requirements Detector 882 determines that prior authorization is required, Prior Authorization Package Generator 886 automatically assembles a prior authorization submission package comprising patient information, provider information, clinical information, supporting clinical documentation, and payer-specific requirements.

Patient information includes patient demographics and identifiers, insurance member ID and group number, subscriber information if patient is a dependent, and patient contact information for payer follow-up.

Provider information includes ordering or referring provider NPI and credentials, rendering or servicing provider NPI if different, facility information if applicable, and provider contact information.

Clinical information includes primary and secondary diagnosis codes (ICD-10-CM) with clinical descriptions, requested procedure codes (CPT, HCPCS) with descriptions, quantity, frequency, and duration of requested services, and date of service or requested service period.

Supporting clinical documentation includes relevant clinical notes extracted from source EHR documentation, diagnostic test results supporting medical necessity, prior treatment history demonstrating step therapy compliance or treatment failure, specialist consultation notes if applicable, and medical necessity narrative generated via LLM summarization of clinical documentation.

Payer-specific requirements include payer-required forms populated with extracted data, attestations and certifications as required, and additional documentation elements specified in payer authorization policy.

Prior Authorization Package Generator 886 uses LLM-based extraction and summarization to populate package elements from source clinical documentation, reducing manual data entry and ensuring consistency between authorization requests and clinical records.

Prior Authorization Submitter 888 transmits the authorization package via the optimal available channel. For payers with electronic prior authorization (EPA) APIs, Prior Authorization Submitter 888 transmits authorization requests via HIPAA X12 278 transactions or payer-proprietary APIs. API submission enables real-time or near-real-time authorization responses for eligible requests.

For payers without direct API integration, Prior Authorization Submitter 888 transmits authorization requests via healthcare clearinghouse partners such as Availity, Waystar, or Change Healthcare that maintain connections to multiple payers. Clearinghouse submission provides broad payer coverage with standardized submission workflows.

For payers requiring portal-based submission, Prior Authorization Submitter 888 may use robotic process automation (RPA) to navigate payer portals, populate required fields, upload documentation, and submit authorization requests. Portal submission is used when API and clearinghouse channels are unavailable.

When automated submission is not available or not appropriate, such as for complex cases requiring clinical discussion or payers requiring phone-based authorization, Prior Authorization Submitter 888 flags the case for staff action with a prepared authorization package, required contact information, and submission instructions.

Prior Authorization Submitter 888 selects the submission channel based on payer channel availability, historical response time by channel, case complexity, and urgency indicators.

Prior Authorization Tracker 889 monitors authorization request status through resolution. For status retrieval, Prior Authorization Tracker 889 polls payer systems via API, clearinghouse, or portal to retrieve authorization status updates including received, in review, approved, denied, and pending additional information statuses.

For alert generation, status changes trigger alerts to appropriate staff. Approval alerts enable scheduling and service rendering. Denial alerts trigger appeal workflows or alternative treatment planning. Pending or additional information alerts prompt staff to gather and submit required documentation. Expiration alerts warn when approved authorizations are approaching expiration dates.

For denial routing, authorization denials are routed to Outcome Feeback System 800 for appeal processing, reusing denial automation capabilities for authorization-related denials.

For authorization data storage, approved authorization numbers, effective dates, expiration dates, and approved service parameters are stored in MAIA Data Store 200 and associated with Medical Claim Data 238 to ensure authorization information is included in subsequent claim submissions.

For claim coordination, Prior Authorization Tracker 889 coordinates with Third-Party Insurance Provider Manager 360 to ensure claims are not submitted for services requiring authorization until authorization is obtained, preventing authorization-related denials.

Prior Authorization Manager 880 may implement Predictive Prior Authorization, wherein authorization requirements are anticipated based on scheduled services, ordered procedures, and clinical context before formal coding. Physician orders trigger authorization requirement lookup. Scheduled procedures trigger proactive authorization initiation. Clinical pathways with known authorization requirements trigger early preparation. Predictive authorization reduces delays between clinical decision and service rendering by initiating authorization workflows as early as possible in the care process.

Prior Authorization Requirements Database 884 may implement Authorization Requirements Inference, wherein prior authorization requirements not explicitly documented are inferred from denial patterns. Claims denied for authorization required but with no documented requirement indicate unstated requirements. Denial patterns by payer, plan, and code combination are statistically analyzed. Inferred requirements are added to the database as soft rules with confidence levels. Inferred requirements are validated when explicit documentation is later obtained. Inference enables proactive authorization even when payer documentation is incomplete or outdated.

Prior Authorization Manager 880 may implement Authorization Approval Prediction, wherein machine learning models estimate the probability of authorization approval based on diagnosis and procedure code combination, patient clinical characteristics, documentation completeness and quality, payer and plan historical approval rates, and prior treatment history including step therapy compliance. Low-probability cases may be flagged for enhanced documentation, peer-to-peer scheduling, or alternative treatment discussion before submission.

Prior Authorization Requirements Database 884 may implement Cross-Payer Aggregation, wherein authorization requirements from multiple sources including clearinghouses, payer APIs, portal extractions, manual entries, and inference are merged and reconciled. Conflicting requirements from different sources are flagged for resolution. Source priority rules determine which source is authoritative when conflicts exist. Temporal versioning tracks requirement changes over time. Gap analysis identifies payer and plan combinations with missing requirement data. Aggregation provides comprehensive authorization intelligence despite fragmented and inconsistent source data.

Prior Authorization Submitter 888 may implement Authorization Urgency Optimization, wherein submission timing and channel selection are optimized based on service urgency. Emergency and urgent services use expedited authorization pathways including phone and concurrent review. Scheduled services use standard pathways with time buffers. Time-sensitive services such as imaging before surgery are prioritized in submission queues. Payer-specific turnaround time data informs scheduling decisions. Urgency optimization ensures authorization workflows align with clinical timelines.

Prior Authorization Manager 880 may implement Clinical Scheduling Integration, wherein authorization workflows are bidirectionally integrated with practice scheduling systems. Scheduled services trigger authorization requirement checks. Authorization status updates trigger scheduling holds or releases. Authorization expiration dates constrain service scheduling windows. Denied authorizations trigger automatic appointment rescheduling or cancellation prompts. Integration prevents scheduling services that cannot be rendered due to authorization status, reducing patient no-shows and staff rework.

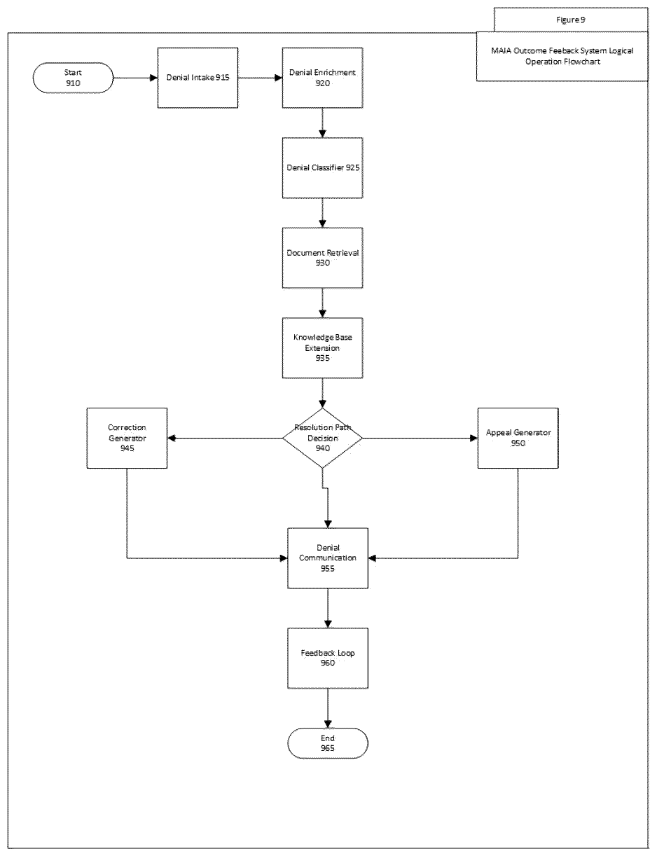

FIG. 9 illustrates a logical operation flowchart of Outcome Feeback System 800.
Step 910: Process Start. The denial automation process is triggered upon receipt of a claim denial from a third-party payer.
Step 915: Denial Intake 830 receives the denial via one or more intake channels: EDI 835 remittance transactions containing CARC (Claim Adjustment Reason Code) and RARC (Remittance Advice Remark Code) data; Payer portal denial notifications with accompanying EOB (Explanation of Benefits) documents; Electronic Remittance Advice (ERA) files; and Scanned paper EOBs requiring OCR processing.
Step 920: Denial Enrichment 832 extracts and enriches denial information: Parsing denial reason codes and remark codes to identify denial category; Extracting denied CPT, ICD-10, and HCPCS codes with associated line-item details; Performing OCR on image-based denial documents to extract structured data; Linking the denial to original Medical Claim Data 238 and source clinical documentation; Retrieving original code justifications and validation results from the coding process; and Gathering historical denial patterns for the same payer, code, and denial reason combination. Denial Intake 830 may implement Denial Intake Normalization, wherein denial data from heterogeneous intake channels is transformed into a standardized internal denial representation. Normalization includes: Mapping payer-specific reason codes to standardized denial taxonomy; Extracting structured fields from unstructured EOB narratives using NLP; Reconciling partial denial data across multiple sources (e.g., ERA amount data combined with portal narrative); and Flagging data quality issues (missing fields, inconsistent amounts, unrecognized codes). Normalized denial records enable consistent downstream processing regardless of intake channel. Denial Enrichment 832 may implement Denial Deduplication, wherein incoming denials are matched against existing denial records to prevent duplicate processing. Deduplication uses: Claim identifier matching (patient, date of service, provider, codes); Fuzzy matching for denials with slight data variations; Timeline validation (same denial received via multiple channels within expected window). Duplicate denials are merged rather than processed independently, preventing wasted effort and conflicting resolution actions.
Step 925: Denial Classifier 834 categorizes the denial into a denial type based on reason codes and enrichment data. Denial types include: Eligibility denials: patient not covered on date of service, coverage terminated, coordination of benefits (COB) issues, subscriber information mismatch; Authorization denials: prior authorization required but not obtained, authorization expired, authorization mismatch (different procedure or provider authorized); Coding/billing denials: invalid or unrecognized code, unbundling edit violation, modifier error or omission, duplicate claim, units exceed limits; Medical necessity denials: LCD (Local Coverage Determination) criteria not met, NCD (National Coverage Determination) criteria not met, diagnosis does not support procedure, experimental/investigational determination; Global period denials: service falls within global surgical period for prior procedure; Bundling denials: service bundled with another billed procedure per NCCI or payer-specific edits; and Administrative denials: timely filing limit exceeded, missing required information, claim format errors, provider enrollment issues. Each denial category is associated with designated resolution pathways optimized for that category type. Denial Classifier 834 may implement Denial Reason Disambiguation, wherein denials with ambiguous or generic reason codes (e.g., “service not covered”) are further analyzed using: Free-text denial narrative parsing via NLP/LLM to identify specific denial reason; Historical pattern matching to similar denials with known root causes; Payer-specific reason code interpretation based on learned payer behavior; and Cross-reference with original claim data to identify likely failure points. Disambiguation enables more accurate classification and targeted resolution strategies when standard reason codes are insufficient. Denial Classifier 834 may implement Multi-Label Classification, wherein a single denial may be assigned multiple denial types when multiple issues contributed to the denial. For example, a denial may be classified as both “coding/billing” (modifier missing) and “medical necessity” (documentation insufficient). Multi-label classification enables comprehensive resolution addressing all contributing factors, rather than partial resolution that results in secondary denials.
Step 930: Document Retrieval 836 automatically gathers appeal packet components based on denial type and payer requirements: Clinical Documentation: Operative reports and procedure notes from EHR; Progress notes documenting medical decision-making; Diagnostic test results (lab, imaging, pathology); Consultation notes from specialists; and Nursing notes and vital signs when relevant.

Administrative Documentation: Prior authorization approval letters and reference numbers; Referral documentation; Patient consent forms; Insurance verification records; and

Original claim submission confirmation.

Payer Forms: Payer-specific appeal forms; Medical necessity determination forms; Peer-to-peer review request forms; and Reconsideration request templates.

Supporting Evidence: Medical literature citations supporting treatment; Professional society guidelines; and Similar case precedents with successful appeals.

Document Retrieval 836 may implement Document Completeness Verification, wherein retrieved documents are validated against denial-type-specific checklists: Medical necessity appeals require clinical documentation meeting coverage criteria; Coding appeals require documentation supporting the billed code level; Authorization appeals require authorization correspondence and timeline evidence. Missing documents are flagged with automated requests to source systems or alerts to Users 102 for manual retrieval. Appeals are not submitted until completeness thresholds are met, reducing appeals returned for insufficient documentation.

Document Retrieval 836 may implement Document Relevance Scoring, wherein retrieved documents are scored for relevance to the specific denial: LLM analysis of document content against denial reason and coverage criteria; Keyword and concept matching between documents and policy requirements; Temporal relevance (documents from date of service vs. historical). High-relevance documents are prioritized in appeal packets; low-relevance documents may be excluded to reduce packet size and reviewer burden. Relevance scores are stored to improve future retrieval.
Step 935: Knowledge Base Extension 837 retrieves payer-specific content relevant to the denial to support resolution:

Payer Medical Policies: Payer coverage policies from policy databases and payer portal documentation; Coverage criteria checklists and required documentation specifications;

Payer-specific coding guidelines and preferences.

Medicare Coverage Data: LCD (Local Coverage Determination) policies by MAC (Medicare Administrative Contractor); NCD (National Coverage Determination) policies; Medicare Benefit Policy Manual references.

Coding Rules: Global period rules by CPT code from CMS Global Service Data; NCCI (CCI) edits including modifier indicators; Payer-specific bundling edits beyond national NCCI; Payer-specific modifier requirements.

Historical Appeal Data: Similar denial appeal outcomes for the target payer; Successful appeal arguments and evidence patterns; Payer-specific response tendencies and timelines.

Knowledge Base Extension 837 may implement Policy Currency Verification, wherein retrieved policies are validated to ensure they were in effect on the date of service: Policies have effective dates and termination dates; Date of service is compared against policy effective period; Superseded policies are flagged to prevent citation of outdated criteria. Policy currency verification prevents appeals based on policies that have been updated or replaced since the service date, improving appeal accuracy and credibility. Knowledge Base Extension 837 may implement Payer Behavior Inference, wherein payer-specific behaviors not explicitly documented in policies are inferred from historical adjudication patterns: Code combinations that are consistently denied despite policy silence; Documentation elements that correlate with approval despite not being required; Seasonal or regional variation in denial patterns; Reviewer-specific patterns when reviewer identifiers are available. Inferred behaviors are stored as soft rules with confidence levels, used to inform resolution strategy and appeal emphasis.
Step 940: Resolution Path Decision 838 determines the optimal resolution strategy based on denial analysis:

Correction and Resubmission is selected when: Denial is due to correctable error (demographics, modifiers, coding typos); Error is clearly identifiable and fixable; Correction addresses the stated denial reason; and Timely filing limits allow resubmission.

Formal Appeal is selected when: Denial disputes coverage, medical necessity, or policy interpretation; Original coding was correct and denial is erroneous; Documentation supports the billed service; Historical appeal success rate for similar denials is acceptable; and Denied amount justifies appeal effort.

Write-Off/No Action may be recommended when: Denial is valid and not correctable;

Appeal success probability is very low; Denied amount does not justify resolution effort; or

Timely filing or appeal deadlines have passed.

Resolution Path Decision 838 may generate a recommendation for Users 102 review or automatically proceed based on configurable automation rules. Resolution Path Decision 838 may implement Expected Value Optimization, wherein each resolution path is evaluated based on: Expected Value=(Probability of Success×Recovery Amount)−Resolution Cost. Factors include: Historical success rates for similar denials by resolution path; Denied amount and potential recovery; Estimated resource cost (automated vs. manual effort, time investment);

Opportunity cost (resources spent on low-probability cases reduce capacity for high-probability cases). Resolution paths are recommended in priority order based on expected value, enabling efficient resource allocation across the denial portfolio.

Resolution Path Decision 838 may implement Multi-Path Resolution, wherein multiple resolution actions are taken in parallel or sequence: Correction and resubmission while simultaneously preparing appeal packet; Initial informal dispute followed by formal appeal if unsuccessful; Multiple appeal levels (first-level appeal, second-level appeal, external review) queued in sequence. Multi-path resolution reduces total resolution time by overlapping preparation and execution stages.
Step 945 (Correction Path): If correction and resubmission is selected, Correction Generator 840 generates corrected claim data. Error Correction 842 applies appropriate corrections based on denial reason: Demographic Corrections: Patient name, date of birth, gender corrections; Subscriber/policy number corrections; Provider NPI or taxonomy corrections; and Place of service corrections.

Coding Corrections: CPT code substitution (correct code for performed procedure);

ICD-10 code correction or addition; HCPCS code corrections.

Modifier Corrections: Adding required modifiers (laterality, distinct service, assistant surgeon); Removing inappropriate modifiers; Correcting modifier sequence.

Quantity/Frequency Corrections: Unit adjustments; Frequency code corrections for recurring services; Date span corrections for multi-day services.

Resubmission Protocol 844 submits the corrected claim with appropriate resubmission indicators: Frequency code 7 (replacement) or 8 (void) per ANSI X12 837 standards; Original claim reference (DCN/ICN); Corrected claim indicator; and Supporting documentation attachments if required.

Correction Generator 840 may implement Correction Confidence Scoring, wherein proposed corrections are assigned confidence levels: High confidence: correction clearly addresses denial reason with strong evidence; Medium confidence: correction is likely but alternative interpretations exist; Low confidence: correction is speculative and may require human review.

High-confidence corrections may be auto-submitted; medium and low-confidence corrections are flagged for human review before resubmission. Confidence scoring prevents resubmission of poorly-founded codes. Correction Generator 840 may implement Correction Impact Simulation, wherein proposed corrections are evaluated against the same validation rules used in original coding: NCCI edit verification on corrected code combinations; Modifier appropriateness validation post-correction; Payment calculation for corrected claim vs. original denied amount; Likelihood of triggering different denial reason. Corrections that would trigger new validation failures are flagged for review. Payment projections help prioritize corrections with significant recovery potential. Corrections that waste adjudication resources and damage payer relationships.
Step 950 (Appeal Path): If formal appeal is selected, Appeal Generator 850 generates an appeal packet. LLM Drafting Protocol 852 uses large language model inference to generate a structured appeal letter comprising:

Header Block: Patient demographics and identifiers; Claim number and date of service; Denied procedure/diagnosis codes; Denial reason code and narrative; Appeal level and deadline.

Reconsideration Request: Clear statement requesting payment reconsideration; Specific relief requested (full payment, partial payment, peer-to-peer review); Regulatory basis for appeal (contractual, regulatory timelines).

Clinical Summary: Patient history relevant to the service; Clinical findings supporting medical necessity; Procedure details with citations to operative notes; Outcomes and clinical rationale.

Policy Analysis: Citation of applicable coverage policy (LCD, NCD, payer policy); Specific policy criteria with section references; Explanation of how criteria are met.

Evidence Mapping: Point-by-point mapping of documentation to each policy criterion; Page/section references to source documents; Highlighting of key clinical evidence.

Conclusion: Summary of argument; Restatement of requested action; Contact information for questions.

The generated appeal letter and supporting documentation are compiled into an appeal packet for submission via appropriate channel (portal, fax, mail, EDI 275). LLM Drafting Protocol 852 may implement Appeal Argument Selection, wherein multiple potential arguments are generated and scored: Medical necessity argument citing clinical evidence; Coding accuracy argument citing documentation support; Policy interpretation argument citing guideline language; Precedent argument citing similar approved claims.

Arguments are scored based on evidence strength, policy alignment, and historical success rate. The strongest arguments are included; weak arguments that might dilute the appeal are omitted. Argument selection optimizes appeal persuasiveness.

LLM Drafting Protocol 852 may implement Payer-Specific Tone Adaptation, wherein appeal language and structure are tailored to payer preferences learned from historical outcomes: Some payers respond better to detailed clinical narratives; Some payers prefer concise bullet-point evidence mapping; Some payers weight literature citations heavily; Some payers respond to regulatory/contractual arguments. Tone adaptation is learned from appeal outcome correlation analysis and continuously refined based on feedback. Appeal Generator 850 may implement Appeal Quality Scoring, wherein generated appeals are evaluated before submission: Completeness: all required sections present; Evidence strength: documentation citations support each claim; Policy alignment: argument addresses stated denial reason; Clarity: argument is logically structured and easy to follow; Compliance: appeal meets payer formatting and submission requirements. Low-quality appeals are flagged for enhancement or human review. Quality scores correlate with appeal success and are used to calibrate generation parameters.
Step 955: Denial Communicator 860 manages multi-channel communication throughout the resolution process:

Voice Agent 862 handles phone-based interactions: Automated status inquiries using IVR navigation and speech recognition; Agent-assisted calls for complex inquiries requiring human payer representatives; Peer-to-peer review scheduling between treating physician and payer medical director; Call outcome documentation with transcription and structured data extraction.

Email Agent 864 handles electronic correspondence: Monitoring incoming email for payer responses, requests for information, and determination notices; Parsing email content to extract status updates, decision outcomes, and action items; Generating follow-up inquiries and information responses; Tracking email threads by denial/appeal case.

Digital Fax Agent 866 is configured to facilitate the transmission of medical claim resolutions, appeal packets, and supporting clinical documentation to a variety of external entities that utilize facsimile communication. This includes the electronic delivery of structured data and generated correspondence to third-party insurance payers, relevant government agencies, and other essential stakeholders involved in the adjudication and review of medical claims. By integrating with the broader communication suite, the agent ensures that information gathered through the Outcome Feedback System 800 is formatted correctly for fax reception, thereby maintaining a consistent flow of information across fragmented administrative channels.

Portal Monitor 868 handles payer portal interactions: API-based integration with payer systems where available; Robotic Process Automation (RPA) for portals without API access;

Status retrieval (appeal received, under review, determination made); Document download (determination letters, EOBs, check images); Documentation upload for appeal supplements.

Status updates from all channels are aggregated and associated with the denial record in MAIA Data Store 200. Status changes trigger workflow events (escalation, follow-up scheduling, feedback processing). Denial Communicator 860 may implement Optimal Channel Selection, wherein the best communication channel is selected for each interaction based on: Interaction type (status check→portal; complex dispute→voice; documentation→email/portal); Payer channel preferences and response patterns; Historical response time by channel; Cost optimization (automated channels preferred when equally effective); Urgency (appeal deadlines may require faster channels). Channel selection is learned from outcome data and continuously optimized.

Denial Communicator 860 may implement Escalation Trigger Detection, wherein communication responses are monitored for escalation opportunities: Payer request for additional information (opportunity to strengthen case); Partial approval indication (opportunity to dispute remaining denial); Peer-to-peer review offer (opportunity for physician advocacy); Regulatory deadline approaching (opportunity to cite compliance requirements); Reviewer assignment (opportunity to research reviewer patterns). Detected triggers generate alerts and recommended actions to maximize resolution success.

Denial Communicator 860 may maintain a Communication Audit Trail comprising: Timestamped log of all communication attempts and outcomes; Recording or transcript of voice interactions (with appropriate consent); Email correspondence archives; Portal interaction screenshots and response captures. Audit trails support dispute resolution when payers claim non-receipt of appeals, provide evidence for regulatory complaints, and enable analysis of communication effectiveness.
Step 960: Feedback Loop 870 implements closed-loop learning from denial outcomes to continuously improve upstream coding accuracy. Medical Billing Code Rules Adjustment 872 applies learnings across system components:

Threshold Adjustments: Tightening confidence thresholds for code families with elevated denial rates; Adjusting payer-specific thresholds based on denial patterns; Modifying complexity routing triggers to send borderline cases to Complex Path.

Validation Rule Enhancement: Adding new validation rules based on observed denial patterns; Refining existing rules to catch previously-missed errors; Creating payer-specific validation rules when denials are payer-specific.

Knowledge Base Updates: Incorporating denial rationale into coding guidance; Adding successful appeal arguments to knowledge base; Updating payer policy representations based on adjudication behavior.

Model Fine-Tuning Signals: Flagging denial cases as negative training examples; Flagging successful corrections as positive examples; Generating training data for model improvement.

Retrieval Optimization: Adjusting retrieval weights to prioritize content that would have prevented denials; Adding new retrieval terms based on denial reason patterns.

Alert Generation: Notifying coding managers of emerging denial patterns; Flagging systemic issues requiring manual intervention; Generating quality reports on denial trends.

Agent Persona Calibration (for Complex Path): Adjusting persona weights based on which perspectives would have predicted denials; Refining debate triggers based on denial correlation.
Step 965: Denial Automation System process ends for the current denial. The denial record is retained in MAIA Data Store 200 for historical analysis, reporting, and ongoing feedback processing. Feedback Loop 870 may implement Denial Root Cause Attribution, wherein each denial is traced back through the coding pipeline to identify the specific failure point:

Documentation Insufficiency: clinical note did not contain required elements—feedback to documentation improvement programs;

Extraction Failure: system failed to extract relevant information present in documentation—feedback to extraction model tuning;

Retrieval Gap: relevant guideline existed but was not retrieved—feedback to retrieval optimization;

Model Error: LLM generated incorrect code despite adequate context and retrieval—feedback to model fine-tuning;

Validation Miss: validation rules failed to catch the error before submission—feedback to rule enhancement; and

External Cause: payer error, coverage change, patient eligibility issue—flagged as non-system issue.

Root cause attribution enables targeted improvement investment rather than blanket changes. Feedback Loop 870 may implement Denial Pattern Anomaly Detection, wherein statistical monitoring identifies unusual denial patterns: Sudden increase in denials for a specific code or code family; New denial reason appearing that hasn't been seen before; Payer-specific denial spike suggesting policy change; Provider-specific denial increase suggesting documentation or coding issue; Geographic variation suggesting MAC or regional payer policy change.

Anomalies trigger alerts for investigation, enabling rapid response to emerging issues before they accumulate significant revenue impact. Feedback Loop 870 may implement Effectiveness Measurement, wherein the impact of feedback-driven changes is tracked: Denial rate change after threshold adjustment; Denial rate change after validation rule addition;

Denial rate change after model fine-tuning; and Time lag between feedback implementation and outcome implementation.

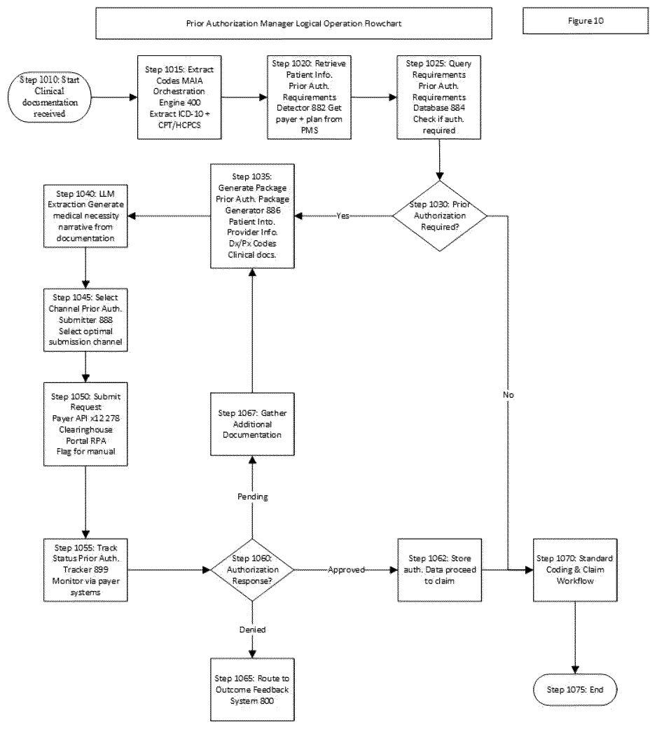

FIG. 10 illustrates a logical operation flowchart of Prior Authorization Manager 880.
Step 1010: Process start. Clinical documentation is received or procedure is scheduled.
Step 1015: MAIA Orchestration Engine 400 extracts diagnosis codes (ICD-10-CM) and procedure codes (CPT, HCPCS) from clinical documentation.
Step 1020: Prior Authorization Requirements Detector 882 retrieves patient payer and plan information from practice management system integration.
Step 1025: Prior Authorization Requirements Detector 882 queries Prior Authorization Requirements Database 884 to determine whether prior authorization is required for the extracted codes under the patient's specific payer and plan.
Step 1030: Decision point. If prior authorization is not required, process proceeds to Step 1070 (standard coding and claim workflow). If prior authorization is required, process proceeds to Step 1035.
Step 1035: Prior Authorization Package Generator 886 assembles authorization submission package including patient information, provider information, diagnosis and procedure codes, and supporting clinical documentation.
Step 1040: LLM-based extraction generates medical necessity narrative from clinical documentation.
Step 1045: Prior Authorization Submitter 888 selects optimal submission channel based on payer capabilities and case characteristics.
Step 1050: Prior Authorization Submitter 888 submits authorization request via payer API, clearinghouse, portal automation, or flags for manual submission.
Step 1055: Prior Authorization Tracker 889 monitors authorization status via periodic polling of payer systems.
Step 1060: Decision point based on authorization response.
Step 1062: If approved, authorization data is stored and process proceeds to Step 1070 (standard coding and claim workflow with authorization reference).
Step 1065: If denied, Authorization denial is routed to Outcome Feeback System 800 for appeal processing or alternative treatment planning.
Step 1067: If pending additional information, process returns to Step 1035 to gather and submit additional documentation.
Step 1070: Standard coding and claim submission workflow proceeds with authorization status incorporated.
Step 1075: Process ends.

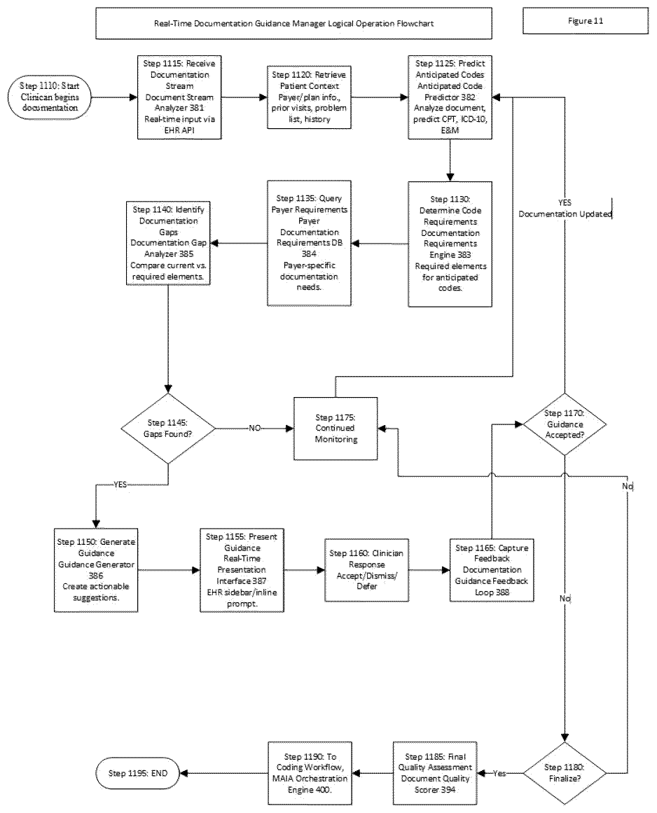

FIG. 11 illustrates a logical operation flowchart of Real-Time Documentation Guidance Manager 380.
Step 1110: Process start. Clinician begins clinical documentation in EHR or documentation system.
Step 1115: Documentation Stream Analyzer 381 receives real-time documentation input via EHR integration API.
Step 1120: Documentation Stream Analyzer 381 retrieves patient context including payer and plan information, prior visit history, and problem list.
Step 1125: Anticipated Code Predictor 382 analyzes documentation in progress to predict likely CPT, HCPCS, ICD-10, and E&M codes.
Step 1130: Documentation Requirements Engine 383 determines documentation elements required to support anticipated codes.
Step 1135: Documentation Requirements Engine 383 queries Payer Documentation Requirements Database 384 to identify payer-specific requirements based on patient's coverage.
Step 1140: Documentation Gap Analyzer 385 compares current documentation against required elements to identify gaps.
Step 1145: Decision point. If no significant gaps are identified, process proceeds to Step 1175 (continue monitoring). If gaps are identified, process proceeds to Step 1150.
Step 1150: Guidance Generator 386 creates actionable guidance for identified gaps, prioritized by revenue impact and denial risk.
Step 1155: Real-Time Presentation Interface 387 delivers guidance to clinician via EHR sidebar, inline prompt, or notification.
Step 1160: Clinician responds to guidance by accepting (incorporating suggested elements), dismissing (with optional reason), or deferring (for later review).
Step 1165: Documentation Guidance Feedback Loop 388 captures clinician response for learning and optimization.
Step 1170: If clinician accepts guidance, documentation is updated. Process returns to Step 1125 to re-analyze with updated documentation.
Step 1175: Continue monitoring. Documentation Stream Analyzer 381 continues receiving documentation updates. Process loops to Step 1125 for continuous analysis.
Step 1180: Decision point. If clinician finalizes and signs documentation, process proceeds to Step 1185. If documentation continues, process loops to Step 1175.
Step 1185: Documentation Quality Scorer 394 generates final quality assessment. Final guidance summary is presented if gaps remain.
Step 1190: Finalized documentation proceeds to standard coding workflow via MAIA Orchestration Engine 400.
Step 1195: Process ends for current documentation session.

1) An Outcome Feedback Computing System comprising at least one processer, coupled to
at least one non-transitory computer readable medium storing instructions, when executed by the at least one processor causes, the at least one processor to execute:
receiving, from a user at an interface operating on a first computing device, user login information;
authenticating, the user login information by matching the user login information with a user account;
receiving, from the user, uploaded medical claim data;
performing, document classification, on the uploaded medical claim data, to determine a document type of the uploaded medical claim data;
intelligently routing, the uploaded medical claim data, to at least one document processing pipelines, being executed by executed by the first computing device or a second computing device, based on the determined document type, of the uploaded medical claim data;
performing, at least one of; feature extraction, data analysis, data modifications or data transformations on the uploaded medical claim data, wherein, the at least one of; feature extraction, data analysis, data modifications or data transformations, is defined by, a set of rules defined for each of the at least one document processing pipeline and for each document type handled by each of the at least one document processing pipeline;
if medical coding is detected during the performing, of at least one of; feature extraction, data analysis, data modifications or data transformations on the uploaded medical claim data;
performing, by at least one code pipeline being executed by the first computing device or the second computing device, or a third computing device, at least one of; feature extraction, data analysis, data modifications or data transformations on the uploaded medical claim data, wherein, the at least one of; feature extraction, data analysis, data modifications or data transformations, is defined by, a set of rules defined for each of the at least one code pipelines and for each code type handled by each of the at least one code pipelines, such as ICD-10, CPT and HCPCS;
inputting, the results of the document processing pipeline into a hybrid data retrieval process, being executed by at least one of the first computing device, the second computing device, or the third computing device, or a fourth computing device, wherein the hybrid data retrieval process uses the results of the document processing pipeline and, if applicable, the results of the code pipeline, to perform a combination of vectorized searching and structured database searching, on at least one knowledge base data storage being executed by at least one of; the first computing device, the second computing device, the third computing device, or the fourth computing device, or a fifth computing device, wherein each of the at least one knowledge base data storage, are specific to each document type and code type;
inputting, the uploaded medical claim data and the results of the hybrid data retrieval process, into at least one embedding model, being executed by at least one of; the first computing device, the second computing device, the third computing device, the fourth computing device, or the fifth computing device, or a sixth computing device, wherein the at least one embedding model is trained to; assign medical billing codes to medical data, determine an initial confidence score for each assigned medical billing code, and generate an initial results summary justification for each assigned medical billing code and corresponding initial confidence score;
comparing, the initial confidence scores to at least one of a plurality of thresholds and triggers:
based on the comparison of initial confidence scores, routing, the uploaded medical claim data and assigned medical billing codes, initial confidence scores and results summary justification, to one of a plurality of validation processing paths, being executed by at least one of; the first computing device, the second computing device, the third computing device, the fourth computing device, the fifth computing device, or the sixth computing device, or a seventh computing device, wherein the plurality of validation paths may include; a fast validation path for assigned billing codes, whose initial confidence scores meet or exceed one of the plurality of thresholds and triggers, and generates at least one data model to determine correlations between the assigned medical billing code and a set of medical data features, and a complex validation path for assigned billing codes, whose initial confidence scores are below one of the plurality of thresholds and triggers and implements crowdsourcing to correlate the assigned medical billing codes with a set of medical data features;
validating, the data model, fast validation path, assigned medical billing codes based on the data model correlations;
validating, the crowdsourced, complex validation path, assigned medical billing codes based on the crowdsourced correlations;
generating, a finalized output which may include, for each of the validated, assigned medical billing codes, assigned to the uploaded medical claim data, the results of the document classification, the results of the document processing pipeline, the results of the hybrid data retrieval process, the relevant knowledge base data, the results of the embedding model, the determined initial confidence scores, the generated initial results summary justification, the comparison of the initial confidence score to the threshold, the results of the validation paths and validation; results of an NCCI validation check, results of an RVU Sequence Validation check, and results of calculating a payment estimate including using RVU data in the estimation and making adjustments to the estimation to account for a geographic region;
presenting, the user with the finalized output at the interface.
inputting, the uploaded medical claim data and the assigned, validated medical billing codes of the uploaded medical claim data, into a third-party insurance provider computing system as a submission of a medical claim;
tracking, the status of the uploaded medical claim data and the assigned validated medical billing codes;
based on the status of the uploaded medical claim data, or one of the assigned and validated medical billing codes, being updated to denied;
for each of the denied, validated medical billing codes;
receiving, a medical claim denial;
automatically generating, and submitting, a resolution of the medical claim denial, based on results of performing at least one of data analytics, data transformations, and feature extractions on the medical claim denial; and
automatically tuning, the assignment, and validation, of the medical billing codes using the results of the performing at least one of data analytics, data modifications, data transformations, and feature extractions on the medical claim denial.

2) The Outcome Feedback Computing System of claim 1, wherein, the at least one non-transitory
computer readable medium further stores instructions, when executed by the at least one processor further causes the at least one processor to execute:
extracting, codes from the medical denial;
enriching, the medical claim denial by performing code lookups on the extracted codes and linking the lookup data to the medical claim denial;
performing, document classification on the medical claim denial, to determine a medical denial type;
linking, knowledge base data related to the medical claim denial, with payer specific content related to the medical claim denial;
determining, a resolution path to handle the medical claim denial;
if the determined resolution path, is correction and resubmission;
correcting, errors, codes and missing data using the enriched data, and
resubmitting, a corrected medical claim;
if the determined resolution path, is appeal;
generating, a structured output, comprising at least one of, a patient/claim identification block, a clear reconsideration request statement, a clinical summary extraction with operative notes, a policy citation with highlighted relevant sections, and an evidence mapping the documentation to the policy criteria; and
submitting, the structured output as an appeal.

3) The Outcome Feedback Computing System of claim 1-2, wherein, the at least one non-transitory computer readable medium further stores instructions, when executed by the at least one processor further causes, the at least one processor to execute:
monitoring, a status of either, the corrected medical claim, or the appeal.

4) The Outcome Feedback Computing System of claims 1-3, wherein, the at least one non-transitory computer readable medium further stores instructions, when executed by the at least one processor further causes, the at least one processor to execute:
in response to monitoring of either the corrected medical claim, or the appeal, detecting, a voice communication; and
implementing, a voice agent to automatically generate and submit a response to the voice communication.

5) The Outcome Feedback Computing System of claims 1-3, wherein, the at least one non-transitory computer readable medium further stores instructions, when executed by the at least one processor further causes, the at least one processor to execute:
in response to monitoring of either the corrected medical claim, or the appeal, detecting, an electronic communication; and
implementing, an email agent to automatically generate and submit a response to the electronic communication.

6) The Outcome Feedback Computing System of claim 1-3, wherein, the at least one non-transitory computer readable medium further stores instructions, when executed by the at least one processor further causes, the at least one processor to execute:
wherein the monitoring of either the corrected medical claim, or the appeal, further comprises using, a portal scraper to actively obtain new information related to the denial; and
providing, a notification in the event of obtaining the new information.

7) The Outcome Feedback Computing System of claim 1, wherein, the at least one non-transitory computer readable medium further stores instructions, when executed by the at least one processor further causes, the at least one processor to execute:
parsing, the medical claim denial to generate tuning data and billing code assignment feedback.

8) The Outcome Feedback Computing System of claims 1 and 7, wherein, the at least one non-transitory computer readable medium further stores instructions, when executed by the at least one processor further causes, the at least one processor to execute:
using, the tuning data and billing code assignment feedback to adjust how medical billing codes are assigned to medical data.

9) A method for implementing a Outcome Feedback Computing System comprising:
receiving, from a user at an interface operating on a first computing device, user login information;
authenticating, the user login information by matching the user login information with a user account;
receiving, from the user, uploaded medical claim data;
performing, document classification, on the uploaded medical claim data, to determine a document type of the uploaded medical claim data;
intelligently routing, the uploaded medical claim data, to at least one document processing pipelines, being executed by executed by the first computing device or a second computing device, based on the determined document type, of the uploaded medical claim data;
performing, at least one of; feature extraction, data analysis, data modifications or data transformations on the uploaded medical claim data, wherein, the at least one of; feature extraction, data analysis, data modifications or data transformations, is defined by, a set of rules defined for each of the at least one document processing pipeline and for each document type handled by each of the at least one document processing pipeline;
if medical coding is detected during the performing, of at least one of; feature extraction, data analysis, data modifications or data transformations on the uploaded medical claim data;
performing, by at least one code pipeline being executed by the first computing device or the second computing device, or a third computing device, at least one of; feature extraction, data analysis, data modifications or data transformations on the uploaded medical claim data, wherein, the at least one of; feature extraction, data analysis, data modifications or data transformations, is defined by, a set of rules defined for each of the at least one code pipelines and for each code type handled by each of the at least one code pipelines, such as ICD-10, CPT and HCPCS;
inputting, the results of the document processing pipeline into a hybrid data retrieval process, being executed by at least one of the first computing device, the second computing device, or the third computing device, or a fourth computing device, wherein the hybrid data retrieval process uses the results of the document processing pipeline and, if applicable, the results of the code pipeline, to perform a combination of vectorized searching and structured database searching, on at least one knowledge base data storage being executed by at least one of; the first computing device, the second computing device, the third computing device, or the fourth computing device, or a fifth computing device, wherein each of the at least one knowledge base data storage, are specific to each document type and code type;
inputting, the uploaded medical claim data and the results of the hybrid data retrieval process, into at least one embedding model, being executed by at least one of; the first computing device, the second computing device, the third computing device, the fourth computing device, or the fifth computing device, or a sixth computing device, wherein the at least one embedding model is trained to; assign medical billing codes to medical data, determine an initial confidence score for each assigned medical billing code, and generate an initial results summary justification for each assigned medical billing code and corresponding initial confidence score;
comparing, the initial confidence scores to at least one of a plurality of thresholds and triggers:
based on the comparison of initial confidence scores, routing, the uploaded medical claim data and assigned medical billing codes, initial confidence scores and results summary justification, to one of a plurality of validation processing paths, being executed by at least one of; the first computing device, the second computing device, the third computing device, the fourth computing device, the fifth computing device, or the sixth computing device, or a seventh computing device, wherein the plurality of validation paths may include; a fast validation path for assigned billing codes, whose initial confidence scores meet or exceed one of the plurality of thresholds and triggers, and generates at least one data model to determine correlations between the assigned medical billing code and a set of medical data features, and a complex validation path for assigned billing codes, whose initial confidence scores are below one of the plurality of thresholds and triggers and implements crowdsourcing to correlate the assigned medical billing codes with a set of medical data features;
validating, the data model, fast validation path, assigned medical billing codes based on the data model correlations;
validating, the crowdsourced, complex validation path, assigned medical billing codes based on the crowdsourced correlations;
generating, a finalized output which may include, for each of the validated, assigned medical billing codes, assigned to the uploaded medical claim data, the results of the document classification, the results of the document processing pipeline, the results of the hybrid data retrieval process, the relevant knowledge base data, the results of the embedding model, the determined initial confidence scores, the generated initial results summary justification, the comparison of the initial confidence score to the threshold, the results of the validation paths and validation; results of an NCCI validation check, results of an RVU Sequence Validation check, and results of calculating a payment estimate including using RVU data in the estimation and making adjustments to the estimation to account for a geographic region;
presenting, the user with the finalized output at the interface.
inputting, the uploaded medical claim data and the assigned, validated medical billing codes of the uploaded medical claim data, into a third-party insurance provider computing system as a submission of a medical claim;
tracking, the status of the uploaded medical claim data and the assigned validated medical billing codes;
based on the status of the uploaded medical claim data, or one of the assigned and validated medical billing codes, being updated to denied;
for each of the denied, validated medical billing codes;
receiving, a medical claim denial;
automatically generating, and submitting, a resolution of the medical claim denial, based on results of performing at least one of data analytics, data transformations, and feature extractions on the medical claim denial; and
automatically tuning, the assignment, and validation, of the medical billing codes using the results of the performing at least one of data analytics, data modifications, data transformations, and feature extractions on the medical claim denial.

10) The method of claim 9 further comprising;
extracting, codes from the medical denial;
enriching, the medical claim denial by performing code lookups on the extracted codes and linking the lookup data to the medical claim denial;
performing, document classification on the medical claim denial, to determine a medical denial type;
linking, knowledge base data related to the medical claim denial, with payer specific content related to the medical claim denial;
determining, a resolution path to handle the medical claim denial;
if the determined resolution path, is correction and resubmission;
correcting, errors, codes and missing data using the enriched data, and
resubmitting, a corrected medical claim;
if the determined resolution path, is appeal;
generating, a structured output, comprising at least one of, a patient/claim identification block, a clear reconsideration request statement, a clinical summary extraction with operative notes, a policy citation with highlighted relevant sections, and an evidence mapping the documentation to the policy criteria; and
submitting, the structured output as an appeal.

11) The method of claims 9-10 further comprising;
monitoring, a status of either, the corrected medical claim, or the appeal.

12) The method of claims 9-11 further comprising;
in response to monitoring of either the corrected medical claim, or the appeal,
detecting, a voice communication; and
implementing, a voice agent to automatically generate and submit a response to the voice communication.

13) The method of claims 9-11 further comprising;
in response to monitoring of either the corrected medical claim, or the appeal, detecting, an electronic communication; and
implementing, an email agent to automatically generate and submit a response to the electronic communication.

14) The method of claims 9-11 further comprising;
wherein the monitoring of either the corrected medical claim, or the appeal, further comprises using, a portal scraper to actively obtain new information related to the denial; and
providing, a notification in the event of obtaining the new information.

15) The method of claim 9 further comprising;
parsing, the medical claim denial to generate tuning data and billing code assignment feedback.

16. The method of claims 9 and 15 further comprising;
using, the tuning data and billing code assignment feedback to adjust how medical billing codes are assigned to medical data.

17. A non-transitory, computer readable medium, comprising at least one processer, coupled to at least one non-transitory computer readable medium storing instructions, when executed by the at least one processor causes, the at least one processor to execute:
receive, from a user at an interface operating on a first computing device, user login information;
authenticate, the user login information by matching the user login information with a user account;
receive, from the user, uploaded medical claim data;
perform, document classification, on the uploaded medical claim data, to determine a document type of the uploaded medical claim data;
intelligently route, the uploaded medical claim data, to at least one document processing pipelines, being executed by executed by the first computing device or a second computing device, based on the determined document type, of the uploaded medical claim data;
perform, at least one of; feature extraction, data analysis, data modifications or data transformations on the uploaded medical claim data, wherein, the at least one of; feature extraction, data analysis, data modifications or data transformations, is defined by, a set of rules defined for each of the at least one document processing pipeline and for each document type handled by each of the at least one document processing pipeline;
if medical coding is detected during the performing, of at least one of; feature extraction, data analysis, data modifications or data transformations on the uploaded medical claim data;
perform, by at least one code pipeline being executed by the first computing device or the second computing device, or a third computing device, at least one of; feature extraction, data analysis, data modifications or data transformations on the uploaded medical claim data, wherein, the at least one of; feature extraction, data analysis, data modifications or data transformations, is defined by, a set of rules defined for each of the at least one code pipelines and for each code type handled by each of the at least one code pipelines, such as ICD-10, CPT and HCPCS;
input, the results of the document processing pipeline into a hybrid data retrieval process, being executed by at least one of the first computing device, the second computing device, or the third computing device, or a fourth computing device, wherein the hybrid data retrieval process uses the results of the document processing pipeline and, if applicable, the results of the code pipeline, to perform a combination of vectorized searching and structured database searching, on at least one knowledge base data storage being executed by at least one of; the first computing device, the second computing device, the third computing device, or the fourth computing device, or a fifth computing device, wherein each of the at least one knowledge base data storage, are specific to each document type and code type;
input, the uploaded medical claim data and the results of the hybrid data retrieval process, into at least one embedding model, being executed by at least one of; the first computing device, the second computing device, the third computing device, the fourth computing device, or the fifth computing device, or a sixth computing device, wherein the at least one embedding model is trained to; assign medical billing codes to medical data, determine an initial confidence score for each assigned medical billing code, and generate an initial results summary justification for each assigned medical billing code and corresponding initial confidence score;
compare, the initial confidence scores to at least one of a plurality of thresholds and triggers:
based on the comparison of initial confidence scores, routing, the uploaded medical claim data and assigned medical billing codes, initial confidence scores and results summary justification, to one of a plurality of validation processing paths, being executed by at least one of; the first computing device, the second computing device, the third computing device, the fourth computing device, the fifth computing device, or the sixth computing device, or a seventh computing device, wherein the plurality of validation paths may include; a fast validation path for assigned billing codes, whose initial confidence scores meet or exceed one of the plurality of thresholds and triggers, and generates at least one data model to determine correlations between the assigned medical billing code and a set of medical data features, and a complex validation path for assigned billing codes, whose initial confidence scores are below one of the plurality of thresholds and triggers and implements crowdsourcing to correlate the assigned medical billing codes with a set of medical data features;
validate, the data model, fast validation path, assigned medical billing codes based on the data model correlations;
validate, the crowdsourced, complex validation path, assigned medical billing codes based on the crowdsourced correlations;
generate, a finalized output which may include, for each of the validated, assigned medical billing codes, assigned to the uploaded medical claim data, the results of the document classification, the results of the document processing pipeline, the results of the hybrid data retrieval process, the relevant knowledge base data, the results of the embedding model, the determined initial confidence scores, the generated initial results summary justification, the comparison of the initial confidence score to the threshold, the results of the validation paths and validation; results of an NCCI validation check, results of an RVU Sequence Validation check, and results of calculating a payment estimate including using RVU data in the estimation and making adjustments to the estimation to account for a geographic region;
present, the user with the finalized output at the interface.
input, the uploaded medical claim data and the assigned, validated medical billing codes of the uploaded medical claim data, into a third-party insurance provider computing system as a submission of a medical claim;
track, the status of the uploaded medical claim data and the assigned validated medical billing codes;
based on the status of the uploaded medical claim data, or one of the assigned and validated medical billing codes, being updated to denied;
for each of the denied, validated medical billing codes;
receive, a medical claim denial;
automatically generate, and submit, a resolution of the medical claim denial, based on results of performing at least one of data analytics, data transformations, and feature extractions on the medical claim denial; and
automatically tune, the assignment, and validation, of the medical billing codes using the results of the performing at least one of data analytics, data modifications, data transformations, and feature extractions on the medical claim denial.

18) The non-transitory, computer readable medium of claim 17, wherein, the at least one non-transitory computer readable medium further stores instructions, when executed by the at least one processor further causes, the at least one processor to execute:
extract, codes from the medical denial;
enrich, the medical claim denial by performing code lookups on the extracted codes and linking the lookup data to the medical claim denial;
perform, document classification on the medical claim denial, to determine a medical denial type;
link, knowledge base data related to the medical claim denial, with payer specific content related to the medical claim denial;
determine, a resolution path to handle the medical claim denial;
if the determined resolution path, is correction and resubmission;
correct, errors, codes and missing data using the enriched data, and
resubmit, a corrected medical claim;
if the determined resolution path, is appeal;
generate, a structured output, comprising at least one of, a patient/claim identification block, a clear reconsideration request statement, a clinical summary extraction with operative notes, a policy citation with highlighted relevant sections, and an evidence mapping the documentation to the policy criteria; and
submit, the structured output as an appeal.

19) The non-transitory, computer readable medium of claim 17, wherein, the at least one non-transitory computer readable medium further stores instructions, when executed by the at least one processor further causes, the at least one processor to execute:
parse, the medical claim denial to generate tuning data and billing code assignment feedback.

20) The non-transitory, computer readable medium of claim 17, wherein, the at least one non-transitory computer readable medium further stores instructions, when executed by the at least one processor further causes, the at least one processor to execute:
use, the tuning data and billing code assignment feedback to adjust how medical billing codes are assigned to medical data.
